(19) United States
(12) Patent Application Publication
(10) Pub. No.: US 2003/0235583 A1
(43) Pub. Date: Dec. 25, 2003

(54) Combined use of a modulator of CD3 and a beta cell resting compound

(76) Inventors: Jeppe Sturis, Vaerlose (DK); Birgitte Koch Michelsen, Lyngby (DK)

Correspondence Address: Reza Green, Esq., Novo Nordisk Pharmaceuticals, Inc., 100 College Road West, Princeton, NJ, 08540, US

(21) Appl. No.: 10/458,900

(22) Filed: Jun. 11, 2003

Related U.S. Application Data
(60) Provisional application No. 60/389,190, filed on Jun. 14, 2002.

(30) Foreign Application Priority Data
Jun. 14, 2002 (DK) PA 2002 00911

(51) Int. Cl. A61K 39/395; A61K 31/549

(52) U.S. Cl. 424/144.1; 514/223.5

(57) ABSTRACT

Methods and uses for the prevention and intervention of Type 1 diabetes and LADA comprising administration of a modulator of CD3 and a beta cell resting compound.

### CROSS REFERENCE TO RELATED APPLICATIONS

This application claims priority under 35 U.S.C. 119 of Danish application PA 2002 00911, filed Jun. 14, 2002, and of U.S. Provisional application No. 60/389,190 filed Jun. 14, 2002, the contents of which are fully incorporated herein by reference.

### FIELD OF THE INVENTION

The present invention relates to methods and uses for the prevention and intervention of Type 1 diabetes and intervention of Latent Autoimmune Diabetes in the Adult (LADA). More specifically, the methods and uses of the invention pertain to administration of a modulator of CD3 in combination with administration of a beta cell resting compound.

### BACKGROUND OF THE INVENTION

Diabetes is a disorder of carbohydrate metabolism characterized by hyperglycemia and glucosuria resulting from insufficient production or utilization of insulin. Diabetes severely affects the quality of life of large parts of the populations in developed countries. Insufficient production of insulin is characterised as Type 1 diabetes and insufficient utilization of insulin is Type 2 diabetes.

Type 1 diabetes mellitus is caused by an autoimmune destruction of the pancreatic beta cells. Likewise, in Latent Autoimmune Diabetes in the Adult (LADA), autoimmunity accelerates the disease process in patients initially diagnosed with Type 2 diabetes, leading to rapid progression to insulin requirement in these patients. T cells play an important role in this process by mediating the autoimmune destruction. It has therefore been hypothesized that it may be possible to influence the development of Type 1 diabetes mellitus as well as the disease progression in LADA patients by regulation of T cells or of the immune system's response to T cells.

CD3 is expressed in T cells. It has been recently demonstrated in humans that short term treatment of new onset Type 1 diabetic patients with an antibody against CD3 is able to attenuate the further destruction of beta-cells, thereby facilitating improved glycemic control of the patients. Ultimately, this gives the patients a better prognosis with respect to the development of diabetic late complications.

Another potential way to intervene in the destruction of beta-cells in the development of Type 1 diabetes is by treatment with compounds that inhibit insulin secretion, thereby inducing beta-cell rest. Bjork, et al. have demonstrated that 3 months treatment with the potassium channel opener diazoxide results in preservation of residual C-peptide, a marker of beta-cell function. We have demonstrated that human islets incubated in high glucose undergo significantly less apoptosis, or programmed cell death, when co-incubated with another potassium channel opener 6-chloro-3-(1-methylcyclopropyl)amino-4H-thieno[3,2-e]-1,2,4-thiadiazine 1,1-dioxide than when not co-incubated with said compound. The glucose induced apoptosis in human beta-cells has been shown to be mediated via the Fas-FasLigand pathway which has been implicated in the development of Type 1 diabetes (Donath Diabetes Aug 2001).

The present invention concerns the combined treatment with at least one compound that regulates CD3 and at least one compound that induces beta-cell rest. The treatment can either be prophylactic, i.e. given to a subject at high risk for the development of Type 1 diabetes, or as an intervention in the disease process at the time it is clinically diagnosed. By this combined treatment, it is possible to avoid the further destruction of beta cells. The treatment can furthermore lead to an increase of beta-cell function as measured by C-peptide after treatment has been discontinued. The effect will be sustained over several years, thereby having a major beneficial impact on the severity of the disease and its complications. Patients will receive state-of-the art therapy with insulin and/or insulin analogs simultaneously during the treatment period in order to provide glycemic control.

In accordance with the present invention, a pharmaceutical combination is provided for use in the prevention and intervention of Type 1 diabetes and LADA, which combination comprises at least one modulator of CD3 and at least one beta cell resting compound.

### SUMMARY OF THE INVENTION

One object of the present invention is to provide methods, which can effectively be used in the in the prevention and intervention of Type 1 diabetes and intervention of LADA.

The invention includes a method for the prevention and intervention of Type 1 diabetes and LADA, which method comprises administration of at least one modulator of CD3 and at least one beta cell resting compound to a patient in need thereof.

The present invention includes the use of at least one modulator of CD3 and at least one beta cell resting compound for the preparation of one ore more medicaments for the prevention and intervention of Type 1 diabetes and LADA in a patient in need thereof.

In one embodiment of the invention, the modulator of CD3 is selected from antibody reactive with CD3 or F(ab′)2 fragment of said antibody.

In another embodiment of the invention the modulator of CD3 is OKT3, hOKT3γ1 (Ala-Ala), 145 2C11 or CAMPATH-3.

In another embodiment of the invention the beta cell resting compound is selected from a potassium channel opener.

In another embodiment of the invention the beta cell resting compound is 6-Chloro-3-(1-methylcyclopropyl)amino-4H-thieno[3,2-e]-1,2,4-thiadiazine 1,1-dioxide. In another embodiment of the invention the modulator of CD3 and the beta cell resting compound are administered in suboptimal dosages.

In yet another embodiment of the invention the modulator of CD3 and the beta cell resting compound are administered in amounts and for a sufficient time to produce a synergistic effect.

Definitions

Co-administration: In the context of the present application, co-administration of two compounds is defined as administration of the two compounds to the patient within one year, including separate administration of two medicaments each containing one of the compounds as well as simultaneous administration whether or not the two compounds are combined in one formulation or whether they are in two separate formulations.

Effective dosage: An effective dosage is a dosage which is sufficient in order for the treatment of the patient to be effective compared with no treatment.

Medicament: Pharmaceutical composition suitable for administration of the pharmaceutically active compound to a patient.

Suboptimal dosage: A suboptimal dosage of a pharmaceutically active compound is a dosage which is below the optimal dosage for that compound when used in single-compound therapy.

Additive effect: An additive effect of two compounds is an effect equal to the sum of the effects of the two individual compounds.

Synergistic effect: A synergistic effect of two compounds is in terms of statistical analysis an effect which is greater than the additive effect which results from the sum of the effects of the two individual compounds.

Favourable effect: A favourable effect of two compounds is in terms of statistical analysis an effect which is greater than the effect of either of the two compounds alone.

Prevention and intervention of Type 1 diabetes and LADA (Latent Autoimmune Diabetes in the Adult): In this application prevention is defined as the management and care of an individual at risk of developing Type 1 diabetes or LADA prior to the clinical onset of the disease. Intervention is defined as the management and care of a Type 1 or LADA diabetes patient at diagnosis or later. The purpose of prevention and intervention is to combat the disease, condition, or disorder and includes the administration of the active compounds to prevent or delay the onset of the symptoms or complications, or alleviating the symptoms or complications, or eliminating the disease, condition, or disorder.

Modulator of CD3: In this application a modulator of CD3 is defined as a compound that interacts with CD3 and modulates the effects of CD3, such as an antibody reactive with CD3.

Beta cell resting compounds: In this application a beta cell resting compound is defined as a compound reducing or inhibiting insulin release, such as potassium channel openers. Specific potassium channel openers of the present invention relates to the compounds of general formula (I) and (Ia) wherein the below definitions of terms is used to describe the compounds.

“Halogen” designates an atom selected from the group consisting of F, Cl, Br and I.

The terms “C₁₋₆-alkyl”, “C₁₋₁₂-alkyl” and “C₁₋₁₈-alkyl” as used herein, alone or in combination, designates a straight or branched, saturated hydrocarbon chain having the indicated number of carbon atoms. Representatives examples include, but are not limited to methyl, ethyl, n-propyl, isopropyl, n-butyl, sec-butyl, isobutyl, tert-butyl, n-pentyl, 2-methylbutyl, 3-methylbutyl, 4-methylpentyl, neopentyl, n-hexyl, 1,2-dimethylpropyl, 2,2-dimethylpropyl, 1,2,2-trimethylpropyl and the like. The term “C₁₋₁₈-alkyl” as used herein also includes secondary C₃₋₆-alkyl and tertiary C₄₋₆-alkyl.

The term “C₁₋₆-alkoxy” as used herein, alone or in combination, refers to a straight or branched monovalent substituent comprising a C₁₋₆-alkyl group linked through an ether oxygen having its free valence bond from the ether oxygen and having 1 to 6 carbon atoms. Representatives groups include, but are not limited to methoxy, ethoxy, propoxy, isopropoxy, butoxy, isobutoxy, sec-butoxy, tert-butoxy, n-pentoxy, neopentoxy, tert-pentoxy, n-hexoxy, isohexoxy and the like.

The term “C₂₋₆-alkenyl” as used herein refers to a straight or branched, unsaturated hydrocarbon chain having 2-6 carbon atoms and one double bond. Examples of such groups include, but are not limited to vinyl, 1-propenyl, 2-propenyl, allyl, isopropenyl, n-butenyl, n-pentenyl, n-hexenyl and the like.

The term “C₂₋₆-alkynyl” as used herein refers to a straight or branched, unsaturated hydrocarbons which contain triple bonds. Examples of such groups include, but are not limited to —C≡—CH, —C≡CCH₃, —CH₂C≡CH, —CH₂CH₂C≡CH, —CH(CH₃)C═CH and the like.

The term “C₁₋₆-alkylthio” as used herein, alone or in combination, refers to a straight or branched monovalent substituent comprising a lower alkyl group linked through a divalent sulfur atom having its free valence bond from the sulfur atom and having 1 to 6 carbon atoms. Representative examples include, but are not limited to, methylthio, ethylthio, n-propylthio, isopropylthio, butylthio, isobutylthio, sec-butylthio, tert-butylthio, n-pentylthio, isopentylthio, neopentylthio, tert-pentylthio, n-hexylthio, isohexyl and the like.

The term “C₃₋₆-cycloalkyl” as used herein refers to a radical of a saturated cyclic hydrocarbon with the indicated number of carbons. Representative examples are cyclopropyl, cyclobutyl, cyclopentyl, cyclohexyl, cycloheptyl, cyclooctyl and the like.

The term “C₁₋₆-alkoxy-C₁₋₆-alkyl” as used herein refers to a group of 2-12 carbon atoms interrupted by an O. Representative examples are CH₂—O—CH₃, CH₂—O—CH₂—CH₃, CH₂—O—CH(CH₃)₂ and the like.

The term “perhalomethyl” means trifluoromethyl, trichloromethyl, tribromomethyl or triiodomethyl.

The term “C₁₋₆-monoalkylamino” as used herein refers to an amino group wherein one of the hydrogen atoms is substituted with a straight or branched, saturated hydrocarbon chain having the indicated number of carbon atoms such as e.g. methylamino, ethylamino, propylamino, n-butylamino, sec-butylamino, isobutylamino, tert-butylamino, n-pentylamino, 2-methylbutylamino, n-hexylamino, 4-methylpentylamino, neopentylamino, n-hexylamino, 2,2-dimethylpropylamino and the like.

The term “C₁₋₆-dialkylamino” as used herein refers to an amino group wherein the two hydrogen atoms independently are substituted with a straight or branched, saturated hydrocarbon chain having the indicated number of carbon atoms; such as dimethylamino, N-ethyl-N-methylamino, diethylamino, dipropylamino, N-(n-butyl)-N-methylamino, di(n-pentyl)amino, and the like.

The term “acyl” as used herein refers to a monovalent substituent comprising a C₁₋₆-alkyl group linked through a carbonyl group; such as e.g. acetyl, propionyl, butyryl, isobutyryl, pivaloyl, valeryl, and the like.

The term “C₁₋₆-alkoxycarbonyl” as used herein refers to a monovalent substituent comprising a C₁₋₆-alkoxy group linked through a carbonyl group; such as e.g. methoxycarbonyl, carbethoxy, propoxycarbonyl, isopropoxycarbonyl, n-butoxycarbonyl, sec-butoxycarbonyl, tert-butoxycarbonyl, 3-methylbutoxycarbonyl, n-hexoxycarbonyl and the like.

The term “3-12 membered mono- or bicyclic system” as used herein refers to a monovalent substituent of formula —NR²R³ or —NR⁸R⁹ where R² and R³, or R³ and R⁹ together with the nitrogen atom form a 3-12 membered mono- or bicyclic system, in which one or more of the carbon atoms may be exchanged with nitrogen, oxygen or sulfur, such as 1-pyrrolidyl, piperidino, morpholino, thiomorpholino, 4-methylpiperazin-1-yl, 7-azabicyclo[2.2.1]heptan-7-yl, tropanyl and the like.

The term “3-6 membered saturated ring system” as used herein refers to a monovalent substituent comprising a monocyclic saturated system containing one or more hetero atoms selected from nitrogen, oxygen and sulfur and having 3-6 members and having its free valence from a carbon atom, e.g. 2-pyrrolidyl, 4-piperidyl, 3-morpholinyl, 1,4-dioxan-2-yl, 5-oxazolidinyl, 4-isoxazolidinyl or 2-thiomorpholinyl.

The term “bicycloalkyl” as used herein refers to a monovalent substituent comprising a bicyclic structure made of 6-12 carbon atoms such as e.g. 2-norbornyl, 7-norbornyl, 2-bicyclo[2.2.2]octyl and 9-bicyclo[3.3.1]nonanyl.

The term “aryl” as used herein refers to phenyl, 1-naphthyl or 2-naphthyl.

The term “heteroaryl” as used herein, alone or in combination, refers to a monovalent substituent comprising a 5-6 membered monocyclic aromatic system or a 9-10 membered bicyclic aromatic system containing one or more heteroatoms selected from nitrogen, oxygen and sulfur, e.g. pyrrole, imidazole, pyrazole, triazole, pyridine, pyrazine, pyrimidine, pyridazine, isothiazole, isoxazole, oxazole, oxadiazole, thiadiazole, quinoline, isoquinoline, quinazoline, quinoxaline, indole, benzimidazole, benzofuran, pteridine and purine.

The term “arylalkyl” as used herein refers to a straight or branched saturated carbon chain containing from 1 to 6 carbons substituted with an aromatic carbohydride; such as benzyl, phenethyl, 3-phenylpropyl, 1-naphtylmethyl, 2-(1-naphtyl)ethyl and the like.

The term “aryloxy” as used herein refers to phenoxy, 1-naphthyloxy or 2-naphthyloxy.

The term “arylalkoxy” as used herein refers to a C₁₋₆-alkoxy group substituted with an aromatic carbohydride, such as benzyloxy, phenethoxy, 3-phenylpropoxy, 1-naphthyl-methoxy, 2-(1-naphtyl)ethoxy and the like.

The term “heteroarylalkyl” as used herein refers to a straight or branched saturated carbon chain containing from 1 to 6 carbons substituted with a heteroaryl group; such as (2-furyl) methyl, (3-furyl)methyl, (2-thienyl)methyl, (3-thienyl)methyl, (2-pyridyl)methyl, 1-methyl-1-(2-pyrimidyl)ethyl and the like.

The term “C₁₋₆-alkylsulfonyl” as used herein refers to a monovalent substituent comprising a C₁₋₆-alkyl group linked through a sulfonyl group such as e.g. methylsulfonyl, ethylsulfonyl, n-propylsulfonyl, isopropylsulfonyl, n-butylsulfonyl, sec-butylsulfonyl, isobutylsulfonyl, tert-butylsulfonyl, n-pentylsulfonyl, 2-methylbutylsulfonyl, 3-methylbutylsulfonyl, n-hexylsulfonyl, 4-methylpentylsulfonyl, neopentylsulfonyl, n-hexylsulfonyl and 2,2-dimethylpropylsulfonyl.

The term “C₁₋₆-monoalkylaminosuffonyl” as used herein refers to a monovalent substituent comprising a C₁₋₆-monoalkylamino group linked through a sulfonyl group such as e.g. methylaminosulfonyl, ethylaminosulfonyl, n-propylaminosulfonyl, isopropylaminosulfonyl, n-butylaminosulfonyl, sec-butylaminosulfonyl, isobutylaminosulfonyl, tert-butylaminosulfonyl, n-pentylaminosulfonyl, 2-methylbutylaminosulfonyl, 3-methylbutylaminosulfonyl, n-hexylaminosulfonyl, 4-methylpentylaminosulfonyl, neopentylaminosulfonyl, n-hexylaminosulfonyl and 2,2-dimethylpropylaminosulfonyl.

The term “C₁₋₆-dialkylaminosulfonyl” as used herein refers to a monovalent substituent comprising a C₁₋₆-dialkylamino group linked through a sulfonyl group such as dimethylaminosulfonyl, N-ethyl-N-methylaminosulfonyl, diethylaminosulfonyl, dipropylaminosulfonyl, N-(n-butyl)-N-methylaminosulfonyl, di(n-pentyl)aminosulfonyl, and the like.

The term “C₁₋₆-alkylsulfinyl” as used herein refers to a monovalent substituent comprising a straight or branched C₁₋₆-alkyl group linked through a sulfinyl group (—S(═O)—); such as e.g. methylsulfinyl, ethylsulfinyl, isopropylsulfinyl, butylsulfinyl, pentylsulfinyl, and the like.

The term “C₁₋₆-alkylcarbonylamino” as used herein refers to an amino group wherein one of the hydrogen atoms is substituted with an acyl group, such as e.g. acetamido, propionamido, isopropylcarbonylamino, and the like.

The term “(C₃₋₄-cycloalkyl)C₁₋₆-alkyl” as used herein, alone or in combination, refers to a straight or branched, saturated hydrocarbon chain having 1 to 6 carbon atoms and being monosubstituted with a C₃₋₆-cycloalkyl group, the cycloalkyl group optionally being mono- or polysubstituted with C₁₋₆-alkyl, halogen, hydroxy or C₁₋₆-alkoxy; such as e.g. cyclopropylmethyl, (1-methylcyclopropyl)methyl, 1-(cyclopropyl)ethyl, cyclopentylmethyl, cyclohexylmethyl, and the like.

The term “arylthio” as used herein, alone or in combination, refers to an aryl group linked through a divalent sulfur atom having its free valence bond from the sulfur atom, the aryl group optionally being mono- or polysubstituted with C₁₋₆-alkyl, halogen, hydroxy or C₁₋₆-alkoxy; e.g. phenylthio, (4-methylphenyl)-thio, (2-chlorophenyl) thio, and the like.

The term “arylsulfinyl” as used herein refers to an aryl group linked through a sulfinyl group (—S(═O)—), the aryl group optionally being mono- or polysubstituted with C₁₋₆-alkyl, halogen, hydroxy or C₁₋₆-alkoxy; such as e.g. phenylsulfinyl, (4-chlorophenyl)sulfinyl, and the like.

The term “arylsulfonyl” as used herein refers to an aryl group linked through a sulfonyl group, the aryl group optionally being mono- or polysubstituted with C₁₋₆-alkyl, halogen, hydroxy or C₁₋₆-alkoxy; such as e.g. phenylsulfonyl, tosyl, and the like.

The term “C₁₋₆-monoalkylaminocarbonyl” as used herein refers to a monovalent substituent comprising a C₁₋₆-monoalkylamino group linked through a carbonyl group such as e.g. methylaminocarbonyl, ethylaminocarbonyl, n-propylaminocarbonyl, isopropylaminocarbonyl, n-butylaminocarbonyl, sec-butylaminocarbonyl, isobutylaminocarbonyl, tert-butylaminocarbonyl, n-pentylaminocarbonyl, 2-methylbutylaminocarbonyl, 3-methylbutylaminocarbonyl, n-hexylaminocarbonyl, 4-methylpentylaminocarbonyl, neopentylaminocarbonyl, n-hexylaminocarbonyl and 2-2-dimethylpropylaminocarbonyl.

The term “C₁-dialkylaminocarbonyl” as used herein refers to a monovalent substituent comprising a C₁₋₆-dialkylamino group linked through a carbonyl group such as dimethylaminocarbonyl, N-ethyl-N-methylaminocarbonyl, diethylaminocarbonyl, dipropylaminocarbonyl, N-(n-butyl)-N-methylaminocarbonyl, di(n-pentyl)aminocarbonyl, and the like.

The term “C₁₋₆-monoalkylaminocarbonylamino” as used herein refers to an amino group wherin one of the hydrogen atoms is substituted with a C₁₋₆-monoalkylaminocarbonyl group, e.g. methylaminocarbonylamino, ethylamino-carbonylamino, n-propylaminocarbonylamino, isopropylaminocarbonylamino, n-butylaminocarbonylamino, secbutylaminocarbonylamino, isobutylaminocarbonylamino, tert-butylaminocarbonylamino, and 2-methylbutylaminocarbonylamino.

The term “C₁₋₆-dialkylaminocarbonylamino” as used herein refers to an amino group wherein one of the hydrogen atoms is substituted with a C₁₋₆-dialkylaminocarbonyl group, such as dimethylaminocarbonylamino, N-ethyl-N-methylaminocarbonylamino, diethylaminocarbonylamino, dipropylaminocarbonylamino, N-(n-butyl)-N-methylaminocarbonylamino, di(n-pentyl)-aminocarbonylamino, and the like.

The term “5- or 6-membered heterocyclic system” as used herein refers to: a monocyclic unsaturated or saturated system containing one, two or three hetero atoms selected from nitrogen, oxygen and sulfur and having 5 members, e.g. pyrrole, furan, thiophene, pyrroline, dihydrofuran, dihydrothiophene, imidazole, imidazoline, pyrazole, pyrazoline, oxazole, thiazole, isoxazole, isothiazole, 1,2,3-oxadiazole, furazan, 1,2,3-triazole, 1,2,3-thiadiazole or 2,1,3-thiadiazole; an aromatic monocyclic system containing one or more nitrogen atoms and having 6 members, e.g. pyridine, pyrazine, pyrimidine, pyridazine, 1,2,4-triazine, 1,2,3-triazine or tetrazine; a non-aromatic monocyclic system containing one or more hetero atoms selected from nitrogen, oxygen and sulfur and having 6 members, e.g. pyran, thiopyran, piperidine, dioxane, oxazine, isoxazine, dithiane, oxathine, thiazine, piperazine, thiadiazine, dithiazine or oxadiazine.

The term “5- or 6-membered nitrogen containing ring” as used herein refers to a monovalent substituent comprising a monocyclic unsaturated or saturated system containing one or more nitrogen atoms and having 5 or 6 members, e.g. pyrrolidinyl, pyrrolinyl, imidazolidinyl, pyrazolidinyl, pyrazolinyl, piperidyl, piperazinyl, pyrrolyl, 2H-pyrrolyl, imidazolyl, pyrazolyl, triazolyl, pyridyl, pyrazinyl, pyrimidinyl, pyridazinyl, morpholino, thiomorpholino, isothiazolyl, isoxazolyl, oxazolyl, oxadiazolyl, thiadiazolyl, 1,3-dioxolanyl and 1,4-dioxolanyl.

The term “4- to 12-membered bicyclic or tricyclic carbocyclic system” as used herein refers to a a monovalent substituent comprising a bicyclic or a tricyclic structure made of 4-12 carbon atoms such as e.g. bicyclo[2.1.1]hexane, bicyclo[2.2.1]heptane, bicyclo [2.2.2]octane, octahydrovpentalene, bicyclo[2.2.0]hexane, adamantane, noradamantane or tricyclo-(4.3.1.1 (3,8))undecane.

### DETAILED DESCRIPTION OF THE INVENTION

It has been discovered that in the prevention and intervention of Type 1 diabetes and LADA, the combined treatment with at least one modulator of CD3 and at least one beta cell resting compound avoids further destruction of beta cells. It has also been discovered that the combined treatment leads to an increase of beta cell function. This increase in beta-cell function may be sustained over several years and gives the patient an improved glycemic control and improved prognosis with respect to microvascular and macrovascular complications.

A synergistic effect of two compounds permits the dosages of these compounds in the combined treatment to be below the optimal dosages of the individual compounds in single-compound treatment. Thus, these suboptimal dosages of the individual compounds reduce side effects since lower dosages are needed for the same therapeutic effect in the combined treatment.

Furthermore, a synergistic effect of the two compounds permits the efficacy of the co-administration of the two compounds to be significantly greater than the sum of the efficacy of each individual compound.

Accordingly, the present invention relates to methods for the prevention and intervention of Type 1 diabetes and LADA, which method comprises administration of a modulator of CD3 and a beta cell resting compound to a patient in need thereof.

The methods comprise administration of an effective amount of a modulator of CD3 and administration of an effective amount of a beta cell resting compound. The two compounds may be co-administered or they may be administered separately as two medicaments. Furthermore, the first compound may be administered in a regimen, which additionally comprises treatment with the second compound.

In one embodiment of the invention, the modulator of CD3 is a CD3 antibody or F(ab′)2 fragment thereof or other CD3 binding compound with same activity.

In another embodiment of the invention, the modulator of CD3 is anti-CD3 monoclonal antibody OKT3 or F(ab′)2 fragment thereof.

In another embodiment of the invention, the modulator of CD3 is anti-CD3 monoclonal antibody hOKT3γ1 (Ala-Ala) or F(ab′)2 fragment thereof.

In another embodiment of the invention the modulator of CD3 is anti-CD3 monoclonal antibody 145 2C11 or F(ab′)2 fragment thereof.

In yet another embodiment of the invention the modulator of CD3 is anti-CD3 monoclonal antibody CAMPATH-3 or F(ab′)2 fragment thereof.

In another embodiment of the invention the beta cell resting compound is a potassium channel opener.

In another embodiment of the invention the beta cell resting compound is a compound selected from the general formula (I):

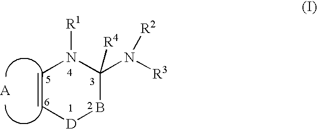

wherein

B represents >NR⁵ or >CR⁵R⁶, wherein R⁵ and R⁶ independently are hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen; or R⁵ and R⁴ together represent one of the bonds in a double bond between the atoms 2 and 3 of formula (I);

D represents —S(═O)₂— or —S(═O)—; or

D-B represents —S(═O)(R⁷)═N—

wherein R⁷ is C₁₋₆-alkyl; or aryl or heteroaryl optionally mono- or poly substituted with halogen, hydroxy, C₁₋₆-alkoxy, aryloxy, arylalkoxy, nitro, amino, C₁₋₆-monoalkyl- or dialkylamino, cyano, acyl, or C₁-alkoxycarbonyl;

R¹ is hydrogen; hydroxy; C₁₋₆-alkoxy; or C₁₋₆-alkyl, C₃₋₆-cycloalkyl, C₂₋₄-alkenyl or C₂₋₆-alkynyl optionally mono- or poly substituted with halogen and R⁴ is hydrogen; or R⁴ together with R⁵ represent one of the bonds in a double bond between the atoms 2 and 3 of formula (I); or R¹ together with R⁴ represent one of the bonds in a double bond between the atoms 3 and 4 of formula (I);

R² is hydrogen; hydroxy; C₁₋₆-alkoxy; or C₁₋₆-alkyl, C₃₋₆-cycloalkyl, C₂₋₆-alkenyl or C₂₋₄-alkynyl optionally mono- or poly substituted with halogen;

R³ is R⁸; —OR⁸; —C(═X)R⁸; —NR⁸R⁹; bicycloalkyl, aryl, heteroaryl, arylalkyl or heteroarylalkyl optionally mono- or poly substituted with halogen, hydroxy, C₁₋₆-alkoxy, aryloxy, arylalkoxy, nitro, amino, C₁₋₆-monoalkyl- or dialkylamino, cyano, oxo, acyl or C₁₋₆-alkoxycarbonyl; or aryl substituted with C₁₋₆-alkyl;

wherein R⁸ is hydrogen; C₃₋₆-cycloalkyl or (C₃₋₄-cycloalkyl)C₁₋₆-alkyl, the C₃₋₆-cycloalkyl group optionally being mono- or poly substituted with C₁₋₆-alkyl, halogen, hydroxy or C₁₋₆-alkoxy; a 3-6 membered saturated ring system comprising one or more nitrogen-, oxygen- or sulfur atoms; or straight or branched C₁₋₁₈-alkyl optionally mono- or poly substituted with halogen, hydroxy, C₁₋₆-alkoxy, C₁₋₆-alkylthio, C₃₋₆-cycloalkyl, aryl, aryloxy, arylalkoxy, nitro, amino, C₁₋₆-monoalkyl- or dialkylamino, cyano, oxo, formyl, acyl, carboxy, C₁₋₆-alkoxycarbonyl, or carbamoyl;

X is O or S;

R⁹ is hydrogen; C₁₋₆-alkyl; C₂₋₄-alkenyl; C₃₋₄-cycloalkyl optionally mono- or poly substituted with C₁₋₆-alkyl, halogen, hydroxy or C₁₋₆-alkoxy; or

R⁸ and R⁹ together with the nitrogen atom form a 3-12 membered mono- or bicyclic system, in which one or more of the carbon atoms may be exchanged with nitrogen, oxygen or sulfur, each of these ring systems optionally being mono- or poly substituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino, oxo; or

R³ is

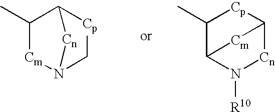

wherein n, m, p independently are 0,1,2,3 and R¹⁰ is hydrogen; hydroxy; C₁₋₆-alkoxy; C₃₋₄-cycloalkyl optionally mono- or poly substituted with C₁₋₆-alkyl, halogen, hydroxy or C₁₋₆-alkoxy; C₁₋₆-alkyl, C₂₋₆-alkenyl or C₂₋₆-alkynyl optionally mono- or poly substituted with halogen; or

R² and R³ together with the nitrogen atom forms a 3-12 membered mono- or bicyclic system, in which one or more of the carbon atoms may be exchanged with nitrogen, oxygen or sulfur, each of these ring systems optionally being mono- or poly substituted with halogen, C₁₋₆ alkyl, hydroxy, C₁₋₆-alkoxy, C₁₋₆-alkoxy-C₁₋₆-alkyl, nitro, amino, cyano, trifluoromethyl, C₁₋₆ monoalkyl- or dialkylamino or oxo;

A together with carbon atoms 5 and 6 of formula (I) represents a 5 or 6 membered heterocyclic system comprising one or more nitrogen-, oxygen- or sulfur atoms, the heterocyclic systems optionally being mono- or poly substituted with halogen; C₁₋₁₂-alkyl; C₃₋₄-cycloalkyl; hydroxy; C₁₋₆-alkoxy; C₁₋₆-alkoxy-C₁₋₆-alkyl; nitro; amino; cyano; cyanomethyl; perhalomethyl; C₁₋₆-monoalkyl- or dialkylamino; sulfamoyl; C₁₋₆-alkylthio; C₁₋₆-alkylsulfonyl; C₁₋₆-alkylsulfinyl; C₁₋₆-alkylcarbonylamino; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl; C₁₋₆-alkoxycarbonyl-C₁₋₆-alkyl; carbamyl; carbamyl-methyl; C₁₋₆-monoalkyl- or dialkylaminocarbonyl; C₁₋₆-monoalkyl- or dialkylaminothiocarbonyl; ureido; C₁₋₆-monoalkyl- or dialkylaminocarbonylamino, thioureido; C₁₋₆-monoalkyl- or dialkylaminothiocarbonyl-amino; C₁₋₆-monoalkyl- or dialkylaminosulfonyl; carboxy; carboxy-C₁₋₆-alkyl; acyl; aryl, arylalkyl, aryloxy, the aryl group optionally being mono- or polysubstituted with C₁₋₆-alkyl, halogen, hydroxy or C₁₋₆-alkoxy; (1,2,4-oxadiazol-5-yl)- or (1,2,4-oxadiazol-3-yl)-C₁₋₆-alkyl the oxadiazolyl group optionally being substituted with C₁₋₆-alkyl or C₃₋₆-cycloalkyl; or a 5-6 membered nitrogen containing ring, optionally substituted with phenyl or C₁₋₆-alkyl; or a pharmaceutically acceptable salt thereof.

Within its scope the invention includes all optical isomers of compounds of the present invention, some of which are optically active, and also their mixtures including racemic mixture thereof.

The scope of the invention also includes all tautomeric forms of the compounds of the present invention as well as metabolites or prodrugs.

A “metabolite” of a compound disclosed in this application is an active derivative of a compound disclosed herein which is produced when the compound is metabolized. Metabolites of compounds disclosed herein can be identified either by administration of a compound to a host and an analysis of blood samples from the host, or by incubation of compounds with hepatic cells in vitro and analysis of the incubant. A “prodrug” is a compound that either is converted into a compound disclosed in the application in vivo or has the same active metabolite as a compound disclosed in this application.

The salts include pharmaceutically acceptable acid addition salts, pharmaceutically acceptable metal salts or optionally alkylated ammonium salts, such as hydrochloric, hydrobromic, hydroiodic, phosphoric, sulfuric, trifluoroacetic, trichloroacetic, oxalic, maleic, pyruvic, malonic, succinic, citric, tartaric, fumaric, mandelic, benzoic, cinnamic, methanesulfonic, ethane sulfonic, picric and the like, and include acids related to the pharmaceutically acceptable salts listed in Journal of Pharmaceutical Science, 66, 2 (1977) and incorporated herein by reference, or lithium, sodium, potassium, magnesium and the like.

In one embodiment of the invention B of formula (I) is >NR⁵ and R⁵ and R⁴ together represent one of the bonds in a double bond between the atoms 2 and 3 of formula (I).

In another embodiment of the invention D is —S(═O)₂—.

In another embodiment of the invention R² is hydrogen or C₁₋₆-alkyl.

In another embodiment of the invention R³ is R⁸, —OR⁸, NR⁸R⁹ or aryl, the aryl groups optionally being substituted with C₁₋₆-alkyl; wherein R³ is hydrogen; C₃₋₄-cycloalkyl; ₆-cycloalkyl)C₁₋₆-alkyl; a 3-6 membered saturated ring system comprising one, two or three nitrogen-, oxygen- or sulfur atoms; or straight or branched C₁₋₁₈-alkyl optionally substituted with halogen, hydroxy, C₁₋₆-alkoxy, C₁₋₆-alkylthio, C₃₋₆-cycloalkyl or aryl, R⁹ is hydrogen, C₁₋₆-alkyl or C₃₋₆-cycloalkyl; or R⁸ and R⁹ together with the nitrogen atom form a 4-6 membered ring.

In another embodiment of the invention wherein R³ is secondary C₃₋₆-alkyl, tertiary C₄₋₆-alkyl, C₃₋₆-cycloalkyl or (C₃₋₆-cycloalkyl)methyl.

In another embodiment of the invention A together with carbon atoms 5 and 6 of formula (I) forms a 5 membered heterocyclic system containing one hetero atom selected from nitrogen and sulfur, the heterocyclic system optionally being mono- or disubstituted with halogen; C₁₋₁₂-alkyl; C₃₋₆-cycloalkyl; cyano; cyanomethyl; perhalomethyl; sulfamoyl; C₁₋₆-alkylthio; C₁₋₆-alkylsulfonyl; C₁₋₆-alkylsulfinyl; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl-C₁₋₆-alkyl; carbamylmethyl; carboxy-C₁₋₆-alkyl; aryloxy; (1,2,4-oxadiazol-5-yl)- or (1,2,4-oxadiazol-3-yl)C₁₋₆-alkyl, the oxadiazolyl group optionally being substituted with C₁₋₆-alkyl or C₃₋₄-cycloalkyl; acyl or a 5-6 membered nitrogen containing ring, optionally substituted with phenyl or C₁₋₆-alkyl.

In another embodiment of the invention A together with carbon atoms 5 and 6 of formula (I) forms a 5 membered heterocyclic system containing two hetero atoms selected from nitrogen, oxygen and sulfur, the heterocyclic system optionally being substituted with halogen; C₁₋₁₂-alkyl; C₃₋₆-cycloalkyl; cyano; cyanomethyl; perhalomethyl; sulfamoyl; C₁₋₆ alkylsulfonyl; C₁₋₆-alkylsulfinyl; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxy-carbonyl-C₁₋₆-alkyl; carbamylmethyl; carboxy-C₁₋₆-alkyl; aryloxy; (1,2,4-oxadiazol-5-yl)- or (1,2,4-oxadiazol-3-yl)C₁₋₆-alkyl, the oxadiazolyl group optionally being substituted with C₁₋₆ alkyl or C₃₋₆-cycloalkyl; acyl; or a 5-6 membered nitrogen containing ring, optionally substituted with phenyl or C₁₋₆-alkyl.

In another embodiment of the invention A together with carbon atoms 5 and 6 of formula (I) forms a 6 membered aromatic heterocyclic system containing one, two or three nitrogen atoms, the heterocyclic system optionally being substituted with halogen; C₁₋₁₂-alkyl; C₃₋₆-cycloalkyl; cyano; cyanomethyl; perhalomethyl; sulfamoyl; C₁₋₆-alkylthio; C₁₋₆alkylsulfonyl; C₁₋₆-alkylsulfinyl; arylthio, arylsulfinyl, arylsulfonyl, the aryll group optionally being mono- or polysubstituted with C₁₋₆-alkyl, halogen, hydroxy or C₁₋₆-alkoxy; C₁₋₆ alkoxycarbonyl-C₁₋₆-alkyl; carbamylmethyl; carboxy-C₁₋₆-alkyl: aryloxy; (1,2,4-oxadiazol-5-yl)- or (1,2,4-oxadiazol-3-yl)C₁₋₆-alkyl, the oxadiazolyl group optionally being substituted with C₁₋₆-alkyl or C₃₋₆-cycloalkyl; acyl; or a 5-6 membered nitrogen containing ring, optionally substituted with phenyl or C₁₋₆-alkyl.

Examples of specific compounds of formula (I) to be used according to this invention are: 6-Chloro-3-(1,2-dimethylpropyl)amino-4H-thieno[3,2-e]-1,2,4-thiadiazine 1,1-dioxide; 6-Chloro-3-ethylamino-4H-thieno[3,2-e]-1,2,4-thiadiazine 1,1-dioxide; 6-Chloro-3-isopropyl-amino-4H-thieno[3,2-e]-1,2,4-thiadiazine 1,1-dioxide; (R)-6-Chloro-3-(1-phenylethyl)amino-4H-thieno[3,2-e]-1,2,4-thiadiazine 1,1-dioxide; 3-Allylamino-6-chloro-4H-thieno[3,2-e]-1,2,4-thiadiazine 1,1-dioxide; 6-Chloro-3-cyclopropylamino-4H-thieno[3,2-e]-1,2,4-thiadiazine 1,1-dioxide; 6-Chloro-3-hexylamino-4H-thieno[3,2-e]-1,2,4-thiadiazine 1,1-dioxide; 6-Chloro-3-tetradecylamino-4H-thieno[3,2-e]-1,2,4-thiadiazine 1,1-dioxide; 6-Chloro-3-methylamino-4H-thieno[3,2,e]-1,2,4-thiadiazine 1,1-dioxide; 3-Benzylamino-6-chloro-4H-thieno[3,2,e]-1,2,4-thiadiazine 1,1-dioxide; 6-Chloro-3-octylamino-4H-thieno[3,2-e]-1,2,4-thiadiazine 1,1-dioxide; 6-Chloro-3-isobutylamino-4H-thieno[3,2-e]-1,2,4-thiadiazine 1,1-dioxide; 6-Chloro-3-(4-phenylbutyl)amino-4H-thieno[3,2-e]-1,2,4-thiadiazine 1,1-dioxide; 6-Chloro-3-(1,5-dimethylhexyl)amino-4H-thieno[3,2-e]-1,2,4-thiadiazine 1,1-dioxide; 6-Chloro-3-propylamino-4H-thieno[3,2-e]-1,2,4-thiadiazine 1,1-dioxide; (R)-6-Chloro-3-(2-hydroxy-1-methylethyl)amino-4H-thieno[3,2-e]-1,2,4-thiadiazine 1,1-dioxide; (S)-6-Chloro-3-(2-hydroxy-1-methylethyl)amino-4H-thieno[3,2-e]-1,2,4-thiadiazine 1,1-dioxide; (R)-3-sec-Butylamino-6-chloro-4H-thieno[3,2-e]-1,2,4-thiadiazine 1,1-dioxide; 3-Butylamino-6-chloro-4H-thieno[3,2-e]-1,2,4-thiadiazine 1,1-dioxide; 3-Isopropylamino-7-methyl-4,7-dihydro-pyrazolo[4,3-e][1,2,4]thiadiazine 1,1-dioxide.

Another example of a specific compound of formula (I) to be used according to this invention is 6-Chloro-3-isopropylamino-4H-thieno[3,2-e]-1,2,4-thiadiazine 1,1-dioxide.

Other examples of specific compounds of formula (I) to be used according to this invention are: 3-Hydrazino-4H-pyrido[4,3-e]-1,2,4-thiadiazine 1,1-dioxide; 3-Benzylamino-4H-pyrido[4,3-e]-1,2,4-thiadiazine 1,1-dioxide; 3-(R)-(1-Phenylethylamino)-4H-pyrido[4,3-e]-1,2,4-thiadiazine 1,1-dioxide; 3-(S)-(1-Phenylethylamino)-4H-pyrido[4,3-e]-1,2,4-thiadiazine 1,1-dioxide; 3-Benzylamino-7-chloro-4H-pyrido[2,3-e]-1,2,4-thiadiazine 1,1-dioxide; 7-Chloro-3-(R)-(1-phenylethylamino)-4H-pyrido[2,3-e]-1,2,4-thiadiazine 1,1-dioxide; 7-Chloro-3-(S)-(1′-phenylethylamino)-4H-pyrido[2,3-e]-1,2,4-thiadiazine 1,1-dioxide; 3-Benzylamino-4H-pyrido[2,3-e]-1,2,4-thiadiazine 1,1-dioxide; 3-(R)-(1-Phenylethylamino)-4H-pyrido[2,3-e]-1,2,4-thiadiazine 1,1-dioxide; 3-(S)-(1-Phenylethylamino)-4H-pyrido[2,3-e]-1,2,4-thiadiazine 1,1-dioxide; 3-(Hexylamino)-4H-pyrido[4,3-e]-1,2,4-thiadiazine 1,1-dioxide; 7-Chloro-3-hexylamino-4H-pyrido[2,3-e]-1,2,4-thiadiazine 1,1-dioxide; 3-Octylamino-4H-pyrido[4,3-e]-1,2,4-thiadiazine 1,1-dioxide; 7-Chloro-3-octylamino-4H-pyrido[2,3-e]-1,2,4-thiadiazine 1,1-dioxide; 3-Allylamino-4H-pyrido[4,3-e]-1,2,4-thiadiazine 1,1-dioxide; 3-Allylamino-7-chloro-4H-pyrido[2,3-e]-1,2,4-thiadiazine 1,1-dioxide; 7-Chloro-3-(2-methoxy-1-methylethyl)amino-4H-pyrido[2,3-e]-1,2,4-thiadiazine 1,1-dioxide; 3-(2-Methoxy-1-methylethyl)amino-4H-pyrido[4,3-e]-1,2,4-thiadiazine 1,1-dioxide; 3-(2-Hydroxy-1-methylethyl)amino-4H-pyrido[4,3-e]-1,2,4-thiadiazine 1,1-dioxide; 3-Benzylamino-2-methyl-2H-pyrido[4,3-e]-1,2,4-thiadiazine 1,1-dioxide; 2-Isopropylamino-3,3-dimethoxy-3H-pyrido[2,3-b][1,4]thiazine 4,4-dioxide.

Other examples of specific compounds of formula (I) to be used according to this invention are: 7-Cyano-3-isopropylamino-6-methyl-4H-thieno[2,3-e]-1,2,4-thiadiazine 1,1-dioxide; 7-Cyano-6-methyl-3-propylamino-4H-thieno[2,3-e]-1,2,4-thiadiazine 1,1-dioxide; 6-Chloro-3-isopentylamino-4H-thieno[3,2-e]-1,2,4-thiadiazine 1,1-dioxide; 6-Chloro-3-(1-methylheptyl)amino-4H-thieno[3,2-e]-1,2,4-thiadiazine 1,1-dioxide; 6-Chloro-3-(1-ethyl-pentyl)amino-4H-thieno[3,2-e]-1,2,4-thiadiazine 1,1-dioxide; 6-Chloro-3-(2-methylbutyl)-amino-4H-thieno[3,2-e]-1,2,4-thiadiazine 1,1-dioxide; 6-Chloro-3-(1-methylhexyl)amino-4H-thieno[3,2-e]-1,2,4-thiadiazine 1,1-dioxide; 6-Chloro-3-cyclopentylamino-4H-thieno[3,2-e]-1,2,4-thiadiazine 1,1-dioxide; 6-Chloro-3-cyclohexylmethylamino-4H-thieno[3,2-e]-1,2,4-thiadiazine 1,1-dioxide; Ethyl 3-(6-chloro-1,4-dihydro-1,1-dioxothieno[3,2-e]-1λ⁶,2,4-thiadiazin-3-ylamino)-butanoate; 3-(6-Chloro-1,4-dihydro-1,1-dioxothieno[3,2-e]-1λ⁶,2,4-thiadiazin-3-ylamino)butanoic acid; 6-Chloro-3-(3-hydroxy-1-methylpropyl)amino-4H-thieno[3,2-e]-1,2,4-thiadiazine 1,1-dioxide; (R)-6-Chloro-3-(1-phenyethyl)amino-4H-thieno[3,2-e]-1,2,4-thiadiazine 1,1-dioxide; (S)-3-sec-Butylamino-6-chloro-4H-thieno[3,2-e]-1,2,4-thiadiazine 1,1-dioxide; 6-Chloro-3-isopropylamino-4H-thieno[2,3-e]-1,2,4-thiadiazine 1,1-dioxide; 6-Chloro-3-cyclopentylamino-4H-thieno[2,3-e]-1,2,4-thiadiazine 1,1-dioxide; 6-Bromo-3-isopropylamino-4H-thieno[3,2-e]-1,2,4-thiadiazine 1,1-dioxide; 3-Isopropylamino-4H-thieno[3,2-e]-1,2,4-thiadiazine 1,1-dioxide; 6-Fluoro-3-isopropylamino-4H-thieno[3,2-e]-1,2,4-thiadiazine 1,1-dioxide; 3-Cyclobutylamino-5,6-dimethyl-4H-thieno[3,2-e]-1,2,4-thiadiazine 1,1-dioxide; 3-Cyclopentylamino-5,6-dimethyl-4H-thieno[3,2-e]-1,2,4-thiadiazine 1,1-dioxide; 3-Isopropylamino-6,7-dimethyl-4H-thieno[2,3-e]-1,2,4-thiadiazine 1,1-dioxide; 3-Cyclobutylamino-6,7-dimethyl-4H-thieno[2,3-e]-1,2,4-thiadiazine 1,1-dioxide; 3-Cyclopentylamino -6,7-dimethyl-4H-thieno[2,3-e]-1,2,4-thiadiazine 1,1-dioxide; 5-Chloro-3-isopropylamino-4H-thieno[3,2-e]-1,2,4-thiadiazine 1,1-dioxide; 5-Chloro-3-propylamino-4H-thieno[3,2-e]-1,2,4-thiadiazine 1,1-dioxide; 5-Chloro-3-cyclopentylamino-4H-thieno[3,2-e]-1,2,4-thiadiazine 1,1-dioxide; 5-Chloro-6-methyl-3-isopropylamino-4H-thieno[3,2-e]-1,2,4-thiadiazine 1,1-dioxide; 6-chloro-3-isopropylamino-5-methyl-4H-thieno[3,2-e]-1,2,4-thiadiazine 1,1-dioxide; 6-chloro-3-cyclopentylamino-5-methyl-4H-thieno[3,2-e]-1,2,4-thiadiazine 1,1-dioxide; 6-Fluoro-3-propylamino-4H-thieno[3,2-e]-1,2,4-thiadiazine 1,1-dioxide; 6-Fluoro-3-cyclopentylamino-4H-thieno[3,2-e]-1,2,4-thiadiazine 1,1-dioxide; 5-Fluoro-3-propylamino-4H-thieno[3,2-e]-1,2,4-thiadiazine 1,1-dioxide; 5-Fluoro-3-isopropylamino-4H-thieno[3,2-e]-1,2,4-thiadiazine 1,1-dioxide; 3-Isopropylamino-7-methyl-4H-thieno[2,3-e]-1,2,4-thiadiazine 1,1-dioxide; 6-Chloro-3-cyclobutylamino-4H-thieno[3,2-e]-1,2,4-thiadiazine 1,1-dioxide; 6-Chloro-3-(2-hydroxyethyl)amino-4H-thieno[3,2-e]-1,2,4-thiadiazine 1,1-dioxide; (±)-3-exo-Bicyclo[2.2.1]hept-2-ylamino-6-chloro-4H-thieno[3,2-e]-1,2,4-thiadiazine 1,1-dioxide; (R)-6-Chloro-3-(2-hydroxypropyl)amino-4H-thieno[3,2-e]-1,2,4-thiadiazine 1,1-dioxide; 6-Bromo-3-isopropylamino-4H-thieno[3,2-e]-1,2,4-thiadiazine 1,1-dioxide; 5,6-Dibromo-3-isopropylamino-4H-thieno[3,2-e]-1,2,4-thiadiazine 1,1-dioxide; 6-Chloro-3-cyclohexylamino-4H-thieno[3,2-e]-1,2,4-thiadiazine 1,1-dioxide; 6-Chloro-3-(furan-2-ylmethyl)amino-4H-thieno[3,2-e]-1,2,4-thiadiazine 1,1-dioxide; 6-Chloro-3-(1-ethylpropyl)-amino-4H-thieno[3,2-e]-1,2,4-thiadiazine 1,1-dioxide; 6-Bromo-3-cyclopentylamino-4H-thieno[3,2-e]-1,2,4-thiadiazine 1,1-dioxide; 6-Chloro-3-(2-methylallyl)amino-4H-thieno[3,2-e]-1,2,4-thiadiazine 1,1-dioxide; 6-Cyano-3-isopropylamino-4H-thieno[3,2-e]-1,2,4-thiadiazine 1,1-dioxide.

In another embodiment of the invention the general formula (I) is selected from

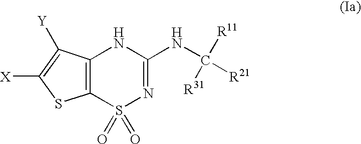

wherein

X and Y independently are hydrogen, halogen, perhalomethyl, C₁₋₆-alkyl or C₁₋₆-alkoxy;

R¹¹, R²¹ and R³¹ independently are C₁₋₆-alkyl, C₂₋₄-alkenyl, C₂₋₆-alkynyl, C₃₋₆-cycloalkyl, carboxy, C₁₋₆-alkoxycarbonyl or aryl, all of which are optionally being mono- or polysubstituted with halogen, hydroxy, oxo, or aryl; or

R¹¹ is as defined above and R²¹—C—R³¹ form a C₃₋₆-cycloalkyl group, optionally being mono- or polysubstituted with C₁₋₆-alkyl, perhalomethyl, halogen, hydroxy or aryl; or

—CR¹¹R²¹R³¹ form a 4- to 12-membered bicyclic or tricyclic carbocyclic system, optionally being mono- or polysubstituted with C₁₋₆-alkyl, perhalomethyl, halogen, hydroxy or aryl; or a salt thereof with a pharmaceutically acceptable acid or base including all optical isomers of compounds of formula (Ia).

In another embodiment of the invention, in formula (Ia) X is halogen and Y is hydrogen.

In another embodiment of the invention, in formula (Ia), X is chloro.

In another embodiment of the invention, in formula (Ia), R¹¹, R²¹ and R³¹ all are C₁₋₆-alkyl.

In another embodiment of the invention, in formula (Ia), R¹¹ is methyl.

In another embodiment of the invention, in formula (Ia), R²¹—C—R³¹ forms a C₃₋₆-cycloalkyl group.

In another embodiment of the invention, in formula (Ia), —CR¹¹R²¹R³¹ forms a tricyclic carbocyclic system.

Examples of specific compounds of formula (Ia) to be used according to this invention are: 3-tert-Butylamino-6-chloro-4H-thieno[3,2-e]-1,2,4-thiadiazine 1,1-dioxide; 6Chloro-3-(1,1-dimethylpropylamino)-4H-thieno[3,2-e]-1,2,4-thiadiazine 1,1-dioxide; 6-Chloro-3-(1-methylcyclopropyl)amino-4H-thieno[3,2-e]-1,2,4-thiadiazine 1,1-dioxide; 6-Chloro-3-(2-hydroxy-1,1-dimethylethylamino)-4H-thieno[3,2-e]-1,2,4-thiadiazine 1,1-dioxide; 6-Chloro-3-(1,1,3,3-tetramethylbutylamino)-4H-thieno[3,2-e]-1,2,4-thiadiazine 1,1-dioxide; 3-(1-Adamantyl)amino-6-chloro-4H-thieno[3,2-e]-1,2,4-thiadiazine 1,1-dioxide; 1-(6-Chloro-1,4-dihydro-1,1-dioxo-thieno[3,2-e]-1λ⁶,2,4-thiadiazin-3-ylamino)-cyclopropanecarboxylic acid ethyl ester; 6-Chloro-3-(1-methyl-1-phenylethyl)amino-4H-thieno[3,2-e]-1,2,4-thiadiazine 1,1-dioxide; 6-Chloro-3-(1-hydroxymethylcyclopentyl)amino-4H-thieno[3,2-e]-1,2,4-thiadiazine 1,1-dioxide; 1-(6-Chloro-1,4-dihydro-1,1-dioxo-thieno[3,2-e]-1λ⁶2,4-thiadiazin-3-ylamino)-cyclopropanecarboxylic acid; 6-Chloro-3-(1-methylcyclobutyl)amino-4H-thieno[3,2-e]-1,2,4-thiadiazine 1,1-dioxide; 6-Chloro-3-(1-methylcyclohexyl)amino-4H-thieno[3,2-e]-1,2,4-thiadiazine 1,1-dioxide; 6-Chloro-3-(1-methylcyclopentyl)amino-4H-thieno[3,2-e]-1,2,4-thiadiazine 1,1-dioxide; 6-Chloro-3-(1-ethylcyclobutyl)amino-4H-thieno[3,2-e]-1,2,4-thiadiazine 1,1-dioxide.

Another example of a specific compound of formula (Ia) to be used according to this invention is 6-Chloro-3-(1-methylcyclopropyl)amino-4H-thieno[3,2-e]-1,2,4-thiadiazine 1,1-dioxide.

Potassium channel openers can readily be determined by those skilled in the art. Methods therefore has been described in e.g. WO 97/26264, WO 97/26265, WO 99/03861, WO 00/37474, and recently reviewed: McClenaghan: Diabetes, Obesitas and Metabolism, 1, 137-150, (1999); Yokoshiki: Am. J. Physiol. 274. C₂₅-C₃₇, (1998); Aguliar-Bryan: Endocrine Reviews, 20, 101-135, (1999).

The compounds of formula (I) and (Ia) of the present invention may be prepared by using the methods taught in e.g. WO 97/26264, WO 97/26265, WO 99/03861 and WO 00/37474, which are hereby incorporated by reference.

In another embodiment of the invention the modulator of CD3 and the beta cell resting compound are co-administered to the patient. The two compounds may be administered as separately formulated compounds or they may be administered as one formulation comprising both compounds.

In a further embodiment, the modulator of CD3 is administered in a regimen, which additionally comprises administration of the beta cell resting compound.

In yet another embodiment, the modulator of CD3 and the beta cell resting compound are administered in suboptimal dosages, i.e. dosages lower than the optimal dosages for single compound therapy.

In a further embodiment the modulator of CD3 and the beta cell resting compound are administered in sufficient amount and for a sufficient time to produce a synergistic effect.

In yet a further embodiment of the invention the modulator of CD3 and the beta cell resting compound are administered in amounts and for a sufficient time to produce an additive effect.

In yet another embodiment of the invention the modulator of CD3 and the beta cell resting compound are administered in amounts and for a sufficient time to produce a favourable effect.

The subject or patient is preferably a mammal, more preferably a human.

The route of administration may be any route, which effectively transports the active compound to the appropriate or desired site of action, such as oral, nasal, buccal, pulmonal, transdermal or parenteral.

Pharmaceutical compositions (or medicaments) containing the modulator of CD3, such as OKY3, hOKT3γ1(Ala-Ala), 145 2C11 or CAM PATH-3, may be administered by suitable dosage forms such as parenteral.

Pharmaceutical compositions (or medicaments) containing the beta cell resting compound, e.g. potassium channel openers of formula (I) and (Ia) of the present invention, may be administered by suitable dosage forms such as oral, nasal, pulmonal, buccal or transdermal to patients in need of such a treatment. The preferred route of administration of said beta cell resting compound is orally. Pharmaceutical compositions containing the beta cell resting compound may be prepared by conventional techniques, e.g. as described in Remington: The Science and Practice of Pharmacy, 19th Edition, Gennaro, Ed., Mack Publishing Co., Easton, Pa., 1995.

Typical compositions of the beta cell resting compounds, e.g. the potassium channel openers of formula (I) and (Ia) of the present invention include a compound of the present invention associated with a pharmaceutically acceptable excipient, which may be a carrier or a diluent or be diluted by a carrier, or enclosed within a carrier, which can be in the form of a capsule, sachet, paper or other container. In making the compositions, conventional techniques for the preparation of pharmaceutical compositions may be used. For example, the active compound will usually be mixed with a carrier, or diluted by a carrier, or enclosed within a carrier, which may be in the form of a ampoule, capsule, sachet, paper, or other container. When the carrier serves as a diluent, it may be solid, semi-solid, or liquid material, which acts as a vehicle, excipient, or medium for the active compound. The active compound can be adsorbed on a granular solid container for example in a sachet. Some examples of suitable carriers are water, salt solutions, alcohol's, polyethylene glycol's, polyhydroxyethoxy-lated castor oil, peanut oil, olive oil, gelatine, lactose, terra alba, sucrose, cyclodextrin, amylose, magnesium stearate, talc, gelatine, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents. The formulations of the invention may be formulated so as to provide quick, sustained, or delayed release of the active ingredient after administration to the patient by employing procedures well known in the art.

The pharmaceutical compositions can be sterilized and mixed, if desired, with auxiliary agents, emulsifiers, salt for influencing osmotic pressure, buffers and/or colouring substances and the like, which do not deleteriously react with the active compound.

If a solid carrier is used for oral administration, the preparation may be tabletted, placed in a hard gelatine capsule in powder or pellet form or it can be in the form of a troche or lozenge. If a liquid carrier is used, the preparation may be in the form of a syrup, emulsion, soft gelatine capsule or sterile injectable liquid such as an aqueous or non-aqueous liquid suspension or solution.

For nasal administration, the preparation may contain the compound of the present invention dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, cornstarch, and/or potato starch. A syrup or elixir can be used in cases where a sweetened vehicle can be employed.

A typical tablet, which may be prepared by conventional tabletting techniques, may contain:

| Core: | | |
|---|---|---|
| Active compound | 5 | mg |
| Colloidal silicon dioxide | 1.5 | mg |
| (Aerosil) | | |
| Cellulose, microcryst. (Avicel) | 70 | mg |
| Modified cellulose gum (Ac-Di-Sol) | 7.5 | mg |
| Magnesium stearate | Ad. | |
| Coating: | | |
| HPMC approx. | 9 | mg |
| *Mywacett 9-40 T approx. | 0.9 | mg |

*Acylated monoglyceride used as plasticizer for film coating.

The beta cell resting compounds are effective over a wide dosage range. For example, in the treatment of adult humans, dosages from 0.1 mg/day to 10000 mg/day, preferably from 10 mg/day to 5000 mg/day may be used. A most preferable dosage is from 100 mg/day to 4000 mg/day. The exact dosage will depend upon the mode of administration, on the therapy desired, the administration form, the subject to be treated and the body weight of the subject to be treated.

Usually, dosage forms suitable for oral, nasal, pulmonary or transdermal administration comprise from about 0.1 mg to about 2000 mg, preferably from about 10 mg to about 1000 mg of the compound of the invention admixed with a pharmaceutically acceptable carrier or diluent.

The treatment with the modulator of CD3 may be repeated at intervals ranging from every 3 months to every 10 years.

The treatment with the beta-cell resting compound may be repeated at intervals ranging from every 3 months to every 10 years.

The treatment with the beta-cell resting compound may be daily for the lifetime of the patient.

Irrespective of the dosage forms for the modulator of CD3 and for the beta cell resting compound, they may advantageously be supplied as a kit for the prevention and intervention of Type 1 diabetes. The kit may contain a single dosage form or it may contain two dosage forms, i.e. one for each compound to be administered.

The combined treatment with a modulator of CD3 and a beta cell resting compound may also be combined with a third or more further pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. Most importantly, when the treatment is used in already diagnosed Type 1 or LADA diabetic patients, co-therapy with insulin, insulin analogues or oral antidiabetic agents will be common. Examples of these pharmacologically active substances are: Insulin, GLP-1 agonists, sulphonylureas, biguanides, meglitinides, glucosidase inhibitors, glucagon antagonists, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents as HMG CoA inhibitors (statins), compounds lowering food intake, RXR agonists and agents acting on the ATP-dependent potassium channel of the β-cells; Cholestyramine, colestipol, clofibrate, gemfibrozil, lovastatin, pravastatin, simvastatin, probucol, dextrothyroxine; β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin; CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β, agonists; histamine H3 antagonists.

It should be understood that any suitable combination of the compounds according to the invention with one or more of the above-mentioned compounds and optionally one or more further pharmacologically active substances are considered to be within the scope of the present invention.

### EXAMPLES

### Example 1

The synergistic effects of the combined use of a modulator of CD3 and a beta cell resting compound can be measured as follows:

Eighty type 1 diabetic patients at diagnosis.

Study Design and Treatment Protocols:

Upon enrollment and following informed consent, patients are be randomized into one of four groups: one receiving anti-CD3 and placebo for a beta cell resting compound, one receiving placebo for anti-CD3 and a beta cell resting compound, one receiving both anti-CD3 and a beta cell resting compound and one receiving placebo for both anti-CD3 and a beta cell resting compound. Anti-CD3 treatment or placebo is administered at a schedule as described by Herold et. al. N Engl J Med 346:1692-98, 2002. Starting the same day as the anti-CD3 treatment, the beta cell resting compound is administered 4 times daily for a period of 3 months. This 3 month period is referred to as the treatment period. Patients receive state-of-the art therapy with insulin and/or insulin analogs simultaneously during the treatment period in order to provide glycemic control.

Endpoints:

The primary endpoint is area under the curve for insulin secretion rates quantified by deconvolution of C-peptide concentrations for the meal test performed after the three month treatment period. Secondary endpoints are fasting C-peptide, insulin secretion rates after the oral glucose tolerance test, use of exogenous insulin, and HbA1c. The statistical analysis is based on baseline subtracted data.

Baseline Assessment and Data Collection

At baseline, subjects or patients have fasting C-peptide, an oral glucose tolerance test and a meal tolerance test performed. Their islet cell antibodies are assessed. The following period, treatment occur. They receive intensive insulin therapy in order to provide glycemic control. They are also receiving (anti-CD3 or placebo) and (the beta cell resting compound or placebo). Between one and seven days after the treatment period has ended and every three months thereafter for an indefinite period, HbA1c fasting C-peptide, oral glucose tolerance tests and meal tests are repeated.

Statistical Analysis

The statistical analysis shows a synergistic effect on the primary endpoint, i.e. the effect of combining anti-CD3 and the beta cell resting compound is greater than the additive effect of either treatment regimen alone. If the effect of administering placebo for anti-CD3 and placebo for the beta cell resting compound is designated A, the effect of administering anti-CD3 and placebo for the beta cell resting compound is designated B, the effect of administering placebo for anti-CD3 and the beta cell resting compound is designated C and the effect of administering anti-CD3 and the beta cell resting compound is designated D, then the statistical analysis shows that D-A is greater than (B-A)+(C-A) with statistical significance at the 0.05 level. The statistical test used is a two-way analysis of variance with anti-CD3 and the beta cell resting compound as the two factors. The interaction term is used to ascertain the presence of synergy.

What is claimed is:

1. A method for the prevention and intervention of Type 1 diabetes and LADA, which method comprises administration of at least one modulator of CD3 and at least one beta cell resting compound to a patient in need thereof.

2. The method according to claim 1, wherein the modulator of CD3 is a CD3 antibody or F(ab′)2 fragment thereof or other CD3 binding compound with same activity.

3. The method according to claim 2, wherein the modulator of CD3 is anti-CD3 mAb OKT3 or F(ab′)2 fragment thereof.

4. The method according to claim 2, wherein the modulator of CD3 is anti-CD3 mAb hOKT3γ1(Ala-Ala) or F(ab′)2 fragment thereof.

5. The method according to claim 2, wherein the modulator of CD3 is anti-CD3 mAb 145 2C11 or F(ab′)2 fragment thereof.

6. The method according to claim 2, wherein the modulator of CD3 is anti-CD3 mAb CAM-PATH-3 or F(ab′)2 fragment thereof.

7. The method according to claim 1 wherein the beta cell resting compound is a potassium channel opener.

8. The method according to claim 7, wherein the beta cell resting compound is a compound of the general formula (I) 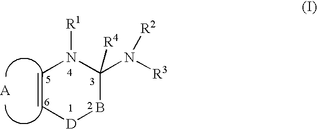
wherein
B represents >NR⁵ or >CR⁵R⁶, wherein R⁵ and R⁶ independently are hydrogen; hydroxy; C₁₋₆ alkoxy; or C₁₋₆-alkyl, C₃₋₆-cycloalkyl, C₂₋₆-alkenyl or C₂₋₆-alkynyl optionally mono- or poly-substituted with halogen; or R⁵ and R⁴ together represent one of the bonds in a double bond between the atoms 2 and 3 of formula (I);
D represents —S(═O)₂— or —S(═O)—; or
D-B represents —S(═O)(R⁷)═N—
wherein R⁷ is C₁₋₆-alkyl; or aryl or heteroaryl optionally mono- or polysubstituted with halogen, hydroxy, C₁₋₆-alkoxy, aryloxy, arylalkoxy, nitro, amino, C₁₋₆-monoalkyl- or dialkylamino, cyano, acyl, or C₁₋₆-alkoxycarbonyl;
R¹ is hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen and R⁴ is hydrogen; or R⁴ together with R⁵ represent one of the bonds in a double bond between the atoms 2 and 3 of formula (I); or R¹ together with R⁴ represent one of the bonds in a double bond between the atoms 3 and 4 of formula (I);
R² is hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen;
R³ is R⁸; —OR⁸; —C(═X)R⁸; —NR⁸R⁹; bicycloalkyl, aryl, heteroaryl, arylalkyl or heteroarylalkyl optionally mono- or poly substituted with halogen, hydroxy, C₁₋₆-alkoxy, aryloxy, arylalkoxy, nitro, amino, C₁₋₆-monoalkyl- or dialkylamino, cyano, oxo, acyl or C₁₋₆-alkoxycarbonyl; or aryl substituted with C₁₆-alkyl;
wherein R⁸ is hydrogen; C₃₋₆-cycloalkyl or (C₃₋₆-cycloalkyl)C₁₋₆-alkyl, the C₃₋₆-cycloalkyl group optionally being mono- or poly substituted with C₁₋₆-alkyl, halogen, hydroxy or C₁₋₆-alkoxy; a 3-6 membered saturated ring system comprising one or more nitrogen-, oxygen- or sulfur atoms; or straight or branched C₁₋₁₈-alkyl optionally mono- or poly substituted with halogen, hydroxy, C₁₋₆-alkoxy, C₁₋₆-alkylthio, C₃₋₆-cycloalkyl, aryl, aryloxy, arylalkoxy, nitro, amino, C₁₋₆-monoalkyl- or dialkylamino, cyano, oxo, formyl, acyl, carboxy, C₁₋₆-alkoxycarbonyl, or carbamoyl;
X is O or S;
R⁹ is hydrogen; C₁₋₆-alkyl; C₂₋₆-alkenyl; C₃₋₆-cycloalkyl optionally mono- or polysubstituted with C₁₋₆-alkyl, halogen, hydroxy or C₁₋₆-alkoxy; or
R⁸ and R⁹ together with the nitrogen atom form a 3-12 membered mono- or bicyclic system, in which one or more of the carbon atoms may be exchanged with nitrogen, oxygen or sulfur, each of these ring systems optionally being mono- or poly substituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁-monoalkyl- or dialkylamino, oxo; or
R³ is 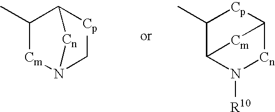
wherein n, m, p independently are 0,1,2,3 and R¹⁰ is hydrogen; hydroxy; C₁₋₆-alkoxy; C₃₋₆-cycloalkyl optionally mono- or poly substituted with C₁₋₆-alkyl, halogen, hydroxy or C₁₋₆ alkoxy; C₁₋₆-alkyl, C₂₋₆-alkenyl or C₂₋₆-alkynyl optionally mono- or polysubstituted with halogen; or
R² and R³ together with the nitrogen atom forms a 3-12 membered mono- or bicyclic system, in which one or more of the carbon atoms may be exchanged with nitrogen, oxygen or sulfur, each of these ring systems optionally being mono- or poly substituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino or oxo;
A together with carbon atoms 5 and 6 of formula (I) represents a 5 or 6 membered hetero-cyclic system comprising one or more nitrogen-, oxygen- or sulfur atoms, the heterocyclic systems optionally being mono- or poly substituted with halogen; C₁₋₁₂-alkyl; C₃₋₄-cycloalkyl; hydroxy; C₁₋₆-alkoxy; C₁₋₆-alkoxy-C₁₋₆-alkyl; nitro; amino; cyano; cyanomethyl; perhalomethyl; C₁₋₆-monoalkyl- or dialkylamino; sulfamoyl; C₁₋₆-alkylthio; C₁₋₆-alkylsulfonyl; C₁₋₆-alkylsulfinyl; C₁₋₆-alkylcarbonylamino; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl; C₁₋₆-alkoxycarbonyl-C₁₋₆-alkyl; carbamyl; carbamyl-methyl; C₁₋₆-monoalkyl- or dialkylamino-carbonyl; C₁₋₆-monoalkyl- or dialkylaminothiocarbonyl; ureido; C₁₋₆-monoalkyl- or dialkylaminocarbonylamino, thioureido; C₁₋₆-monoalkyl- or dialkylaminothiocarbonyl-amino; C₁₋₆ monoalkyl- or dialkylaminosulfonyl; carboxy; carboxy-C₁₋₆-alkyl; acyl; aryl, arylalkyl, aryloxy, the aryl group optionally being mono- or polysubstituted with C₁₋₆-alkyl, halogen, hydroxy or C₁₋₆-alkoxy; (1,2,4-oxadiazol-5-yl)- or (1,2,4-oxadiazol-3-yl)-C₁₋₆-alkyl the oxadiazolyl group optionally being substituted with C₁₋₆-alkyl or C₃₋₄-cycloalkyl; or a 5-6 membered nitrogen containing ring, optionally substituted with phenyl or C₁₋₆-alkyl; or
a salt thereof with a pharmaceutically acceptable acid or base, optical isomers, mixtures, racemic mixtures, or any tautomeric form thereof.

9. The method according to claim 8, wherein the compound of formula (I) is selected from the group:
6-Chloro-3-(1,2-dimethylpropyl)amino-4H-thieno[3,2-e]-1,2,4-thiadiazine 1,1-dioxide;
6-Chloro-3-ethylamino-4H-thieno[3,2-e]-1,2,4-thiadiazine 1,1-dioxide;
6-Chloro-3-isopropylamino-4H-thieno[3,2-e]-1,2,4-thiadiazine 1,1-dioxide;
(R)-6-Chloro-3-(1-phenylethyl)amino-4H-thieno[3,2-e]-1,2,4-thiadiazine 1,1-dioxide;
3-Allylamino-6-chloro-4H-thieno[3,2-e]-1,2,4-thiadiazine 1,1-dioxide;
6-Chloro-3-cyclopropylamino-4H-thieno[3,2-e]-1,2,4-thiadiazine 1,1-dioxide;
6-Chloro-3-hexylamino-4H-thieno[3,2-e]-1,2,4-thiadiazine 1,1-dioxide;
6-Chloro-3-tetradecylamino-4H-thieno[3,2-e]-1,2,4-thiadiazine 1,1-dioxide;
6-Chloro-3-methylamino-4H-thieno[3,2,e]-1,2,4-thiadiazine 1,1-dioxide;
3-Benzylamino-6-chloro-4H-thieno[3,2,e]-1,2,4-thiadiazine 1,1-dioxide;
6-Chloro-3-octylamino-4H-thieno[3,2-e]-1,2,4-thiadiazine 1,1-dioxide;
6-Chloro-3-isobutylamino-4H-thieno[3,2-e]-1,2,4-thiadiazine 1,1-dioxide;
6-Chloro-3-(4-phenylbutyl)amino-4H-thieno[3,2-e]-1,2,4-thiadiazine 1,1-dioxide;
6-Chloro-3-(1,5-dimethylhexyl)amino-4H-thieno[3,2-e]-1,2,4-thiadiazine 1,1-dioxide;
6-Chloro-3-propylamino-4H-thieno[3,2-e]-1,2,4-thiadiazine 1,1-dioxide;
(R)-6-Chloro-3-(2-hydroxy-1-methylethyl)amino-4H-thieno[3,2-e]-1,2,4-thiadiazine 1,1-dioxide;
(S)-6-Chloro-3-(2-hydroxy-1-methylethyl)amino-4H-thieno[3,2-e]-1,2,4-thiadiazine 1,1-dioxide;
(R)-3-sec-Butylamino-6-chloro-4H-thieno[3,2-e]-1,2,4-thiadiazine 1,1-dioxide;
3-Butylamino-6-chloro-4H-thieno[3,2-e]-1,2,4-thiadiazine 1,1-dioxide; and
3-Isopropylamino-7-methyl-4,7-dihydro-pyrazolo[4,3-e][1,2,4]thiadiazine 1,1-dioxide; or
a salt thereof with a pharmaceutically acceptable acid or base, optical isomers, mixtures, racemic mixtures, or any tautomeric form thereof.

10. The method according to claim 8, wherein the compound of formula (I) is selected from the group:
3-Hydrazino-4H-pyrido[4,3-e]-1,2,4-thiadiazine 1,1-dioxide;
3-Benzylamino-4H-pyrido[4,3-e]-1,2,4-thiadiazine 1,1-dioxide;
3-(R)-(l-Phenylethylamino)-4H-pyrido[4,3-e]-1,2,4-thiadiazine 1,1-dioxide;
3-(S)-(1-Phenylethylamino)4H-pyrido[4,3-e]-1,2,4-thiadiazine 1,1-dioxide;
3-Benzylamino-7-chloro-4H-pyrido[2,3-e]-1,2,4-thiadiazine 1,1-dioxide;
7-Chloro-3-(R)-(1-phenylethylamino)-4H-pyrido[2,3-e]-1,2,4-thiadiazine 1,1-dioxide;
7-Chloro-3-(S)-(1′-phenylethylamino)-4H-pyrido[2,3-e]-1,2,4-thiadiazine 1,1-dioxide;
3-Benzylamino-4H-pyrido[2,3-e]-1,2,4-thiadiazine 1,1-dioxide;
3-(R)-(1-Phenylethylamino)-4H-pyrido[2,3-e]-1,2,4-thiadiazine 1,1-dioxide;
3-(S)-(1-Phenylethylamino)-4H-pyrido[2,3-e]-1,2,4-thiadiazine 1,1-dioxide;
3-(Hexylamino)-4H-pyrido[4,3-e]-1,2,4-thiadiazine 1,1-dioxide;
7-Chloro-3-hexylamino-4H-pyrido[2,3-e]-1,2,4-thiadiazine 1,1-dioxide;
3-Octylamino-4H-pyrido[4,3-e]-1,2,4-thiadiazine 1,1-dioxide;
7-Chloro-3-octylamino-4H-pyrido[2,3-e]-1,2,4-thiadiazine 1,1-dioxide;
3-Allylamino-4H-pyrido[4,3-e]-1,2,4-thiadiazine 1,1-dioxide;
3-Allylamino-7-chloro-4H-pyrido[2,3-e]-1,2,4-thiadiazine 1,1-dioxide;
7-Chloro-3-(2-methoxy-1-methylethyl)amino-4H-pyrido[2,3-e]-1,2,4-thiadiazine 1,1-dioxide;
3-(2-Methoxy-1-methylethyl)amino-4H-pyrido[4,3-e]-1,2,4-thiadiazine 1,1-dioxide;
3-(2-Hydroxy-1-methylethyl)amino-4H-pyrido[4,3-e]-1,2,4-thiadiazine 1,1-dioxide;
3-Benzylamino-2-methyl-2H-pyrido[4,3-e]-1,2,4-thiadiazine 1,1-dioxide; and
2-Isopropylamino-3,3-dimethoxy-3H-pyrido[2,3-b][1,4]thiazine 4,4-dioxide; or
a salt thereof with a pharmaceutically acceptable acid or base, optical isomers, mixtures, racemic mixtures, or any tautomeric form thereof.

11. The method according to claim 8, wherein the compound of formula (I) is selected from the group:
7-Cyano-3-isopropylamino-6-methyl-4H-thieno[2,3-e]-1,2,4-thiadiazine 1,1-dioxide;
7-Cyano-6-methyl-3-propylamino-4H-thieno[2,3-e]-1,2,4-thiadiazine 1,1-dioxide;
6-Chloro-3-isopentylamino-4H-thieno[3,2-e]-1,2,4-thiadiazine 1,1-dioxide;
6-Chloro-3-(1-methylheptyl)amino-4H-thieno[3,2-e]-1,2,4-thiadiazine 1,1-dioxide;
6-Chloro-3-(1-ethylpentyl)amino-4H-thieno[3,2-e]-1,2,4-thiadiazine 1,1-dioxide;
6-Chloro-3-(2-methylbutyl)amino-4H-thieno[3,2-e]-1,2,4-thiadiazine 1,1-dioxide;
6-Chloro-3-(l-methylhexyl)amino-4H-thieno[3,2-e]-1,2,4-thiadiazine 1,1-dioxide;
6-Chloro-3-cyclopentylamino-4H-thieno[3,2-e]-1,2,4-thiadiazine 1,1-dioxide;
6-Chloro-3-cyclohexylmethylamino-4H-thieno[3,2-e]-1,2,4-thiadiazine 1,1-dioxide;
Ethyl 3-(6-chloro-1,4-dihydro-1,1-dioxothieno[3,2-e]-1λ⁶,2,4-thiadiazin-3-ylamino)-butanoate;
3-(6-Chloro-1,4-dihydro-1,1-dioxothieno[3,2-e]-1λ⁶,2,4-thiadiazin-3-ylamino)butanoic acid;
6-Chloro-3-(3-hydroxy-1-methylpropyl)amino-4H-thieno[3,2-e]-1,2,4-thiadiazine 1,1-dioxide;
(R)-6-Chloro-3-(1-phenylethyl)amino-4H-thieno[3,2-e]-1,2,4-thiadiazine 1,1-dioxide;
(S)-3-sec-Butylamino-6-chloro-4H-thieno[3,2-e]-1,2,4-thiadiazine 1,1-dioxide;
6-Chloro-3-isopropylamino-4H-thieno[2,3-e]-1,2,4-thiadiazine 1,1-dioxide;
6-Chloro-3-cyclopentylamino-4H-thieno[2,3-e]-1,2,4-thiadiazine 1,1-dioxide;
6-Bromo-3-isopropylamino-4H-thieno[3,2-e]-1,2,4-thiadiazine 1,1-dioxide;
3-Isopropylamino-4H-thieno[3,2-e]-1,2,4-thiadiazine 1,1-dioxide;
6-Fluoro-3-isopropylamino-4H-thieno[3,2-e]-1,2,4-thiadiazine 1,1-dioxide;
3-Cyclobutylamino-5,6-dimethyl-4H-thieno[3,2-e]-1,2,4-thiadiazine 1,1-dioxide;
3-Cyclopentylamino-5,6-dimethyl-4H-thieno[3,2-e]-1,2,4-thiadiazine 1,1-dioxide;
3-Isopropylamino-6,7-dimethyl-4H-thieno[2,3-e]-1,2,4-thiadiazine 1,1-dioxide;
3-Cyclobutylamino-6,7-dimethyl-4H-thieno[2,3-e]-1,2,4-thiadiazine 1,1-dioxide;
3-Cyclopentylamino-6,7-dimethyl-4H-thieno[2,3-e]-1,2,4-thiadiazine 1,1-dioxide;
5-Chloro-3-isopropylamino-4H-thieno[3,2-e]-1,2,4-thiadiazine 1,1-dioxide;
5-Chloro-3-propylamino-4H-thieno[3,2-e]-1,2,4-thiadiazine 1,1-dioxide;
5-Chloro-3-cyclopentylamino-4H-thieno[3,2-e]-1,2,4-thiadiazine 1,1-dioxide;
5-Chloro-6-methyl-3-isopropylamino-4H-thieno[3,2-e]-1,2,4-thiadiazine 1,1-dioxide;
6-chloro-3-isopropylamino-5-methyl-4H-thieno[3,2-e]-1,2,4-thiadiazine 1,1-dioxide;
6-chloro-3-cyclopentylamino-5-methyl-4H-thieno[3,2-e]-1,2,4-thiadiazine 1,1-dioxide;
6-Fluoro-3-propylamino-4H-thieno[3,2-e]-1,2,4-thiadiazine 1,1-dioxide;
6-Fluoro-3-cyclopentylamino-4H-thieno[3,2-e]-1,2,4-thiadiazine 1,1-dioxide;
5-Fluoro-3-propylamino-4H-thieno[3,2-e]-1,2,4-thiadiazine 1,1-dioxide;
5-Fluoro-3-isopropylamino-4H-thieno[3,2-e]-1,2,4-thiadiazine 1,1-dioxide;
3-Isopropylamino-7-methyl-4H-thieno[2,3-e]-1,2,4-thiadiazine 1,1-dioxide;
6-Chloro-3-cyclobutylamino-4H-thieno[3,2-e]-1,2,4-thiadiazine 1,1-dioxide;
6-Chloro-3-(2-hydroxyethyl)amino-4H-thieno[3,2-e]-1,2,4-thiadiazine 1,1-dioxide;
(±)-3-exo-Bicyclo[2.2.1]hept-2-ylamino-6-chloro-4H-thieno[3,2-e]-1,2,4-thiadiazine 1,1-dioxide;
(R)-6-Chloro-3-(2-hydroxypropyl)amino-4H-thieno[3,2-e]-1,2,4-thiadiazine 1,1-dioxide;
6-Bromo-3-isopropylamino-4H-thieno[3,2-e]-1,2,4-thiadiazine 1,1-dioxide;
5,6-Dibromo-3-isopropylamino-4H-thieno[3,2-e]-1,2,4-thiadiazine 1,1-dioxide;
6-Chloro-3-cyclohexylamino-4H-thieno[3,2-e]-1,2,4-thiadiazine 1,1-dioxide;
6-Chloro-3-(furan-2-ylmethyl)amino-4H-thieno[3,2-e]-1,2,4-thiadiazine 1,1-dioxide;
6-Chloro-3-(1-ethylpropyl)amino-4H-thieno[3,2-e]-1,2,4-thiadiazine 1,1-dioxide;
6-Bromo-3-cyclopentylamino-4H-thieno[3,2-e]-1,2,4-thiadiazine 1,1-dioxide;
6-Chloro-3-(2-methylallyl)amino-4H-thieno[3,2-e]-1,2,4-thiadiazine 1,1-dioxide; and
6-Cyano-3-isopropylamino-4H-thieno[3,2-e]-1,2,4-thiadiazine 1,1-dioxide; or
a salt thereof with a pharmaceutically acceptable acid or base, optical isomers, mixtures, racemic mixtures, or any tautomeric form thereof.

12. The method according to claim 8, wherein the general formula (I) is 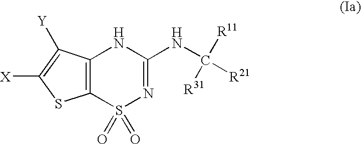
wherein
X and Y independently are hydrogen, halogen, perhalomethyl, C₁₋₆-alkyl or C₁₋₆-alkoxy;
R¹¹, R²¹ and R³¹ independently are C₁₋₆-alkyl, C₂₋₆-alkenyl, C₂₋₆-alkynyl, C₃₋₆-cycloalkyl, carboxy, C₁₋₆-alkoxycarbonyl or aryl, all of which are optionally being mono- or polysubstituted with halogen, hydroxy, oxo, or aryl; or
R¹¹ is as defined above and R²¹—C—R³¹ form a C₃₋₆-cycloalkyl group, optionally being mono- or polysubstituted with C₁₋₆-alkyl, perhalomethyl, halogen, hydroxy or aryl; or
—CR¹¹R²¹R³¹ form a 4- to 12-membered bicyclic or tricyclic carbocyclic system, optionally being mono- or polysubstituted with C₁₋₆-alkyl, perhalomethyl, halogen, hydroxy or aryl; or
a salt thereof with a pharmaceutically acceptable acid or base, optical isomers, mixtures, racemic mixtures, or any tautomeric form thereof.

13. The method according to claim 12, wherein the compound of formula (Ia) is selected from the group:
3-tert-Butylamino-6-chloro-4H-thieno[3,2-e]-1,2,4-thiadiazine 1,1-dioxide;
6-Chloro-3-(1,1-dimethylpropylamino)-4H-thieno[3,2-e]-1,2,4-thiadiazine 1,1-dioxide;
6-Chloro-3-(1-methylcyclopropyl)amino-4H-thieno[3,2-e]-1,2,4-thiadiazine 1,1-dioxide;
6-Chloro-3-(2-hydroxy-1,1-dimethylethylamino)-4H-thieno[3,2-e]-1,2,4-thiadiazine 1,1-dioxide;
6-Chloro-3-(1,1,3,3-tetramethylbutylamino)-4H-thieno[3,2-e]-1,2,4-thiadiazine 1,1-dioxide;
3-(1-Adamantyl)amino-6-chloro-4H-thieno[3,2-e]-1,2,4-thiadiazine 1,1-dioxide;
1-(6-Chloro-1,4-dihydro-1,1-dioxo-thieno[3,2-e]-1λ⁶,2,4-thiadiazin-3-ylamino)-cyclopropane-carboxylic acid ethyl ester;
6-Chloro-3-(1-methyl-1-phenylethyl)amino-4H-thieno[3,2-e]-1,2,4-thiadiazine 1,1-dioxide;
6-Chloro-3-(1-hydroxymethylcyclopentyl)amino-4H-thieno[3,2-e]-1,2,4-thiadiazine 1,1-dioxide;
1-(6-Chloro-1,4-dihydro-1,1-dioxo-thieno[3,2-e]-1λ⁶,2,4-thiadiazin-3-ylamino)-cyclopropane-carboxylic acid;
6-Chloro-3-(1-methylcyclobutyl)amino-4H-thieno[3,2-e]-1,2,4-thiadiazine 1,1-dioxide;
6-Chloro-3-(1-methylcyclohexyl)amino-4H-thieno[3,2-e]-1,2,4-thiadiazine 1,1-dioxide;
6-Chloro-3-(1-methylcyclopentyl)amino-4H-thieno[3,2-e]-1,2,4-thiadiazine 1,1-dioxide; and
6-Chloro-3-(1-ethylcyclobutyl)amino-4H-thieno[3,2-e]-1,2,4-thiadiazine 1,1-dioxide; or
a salt thereof with a pharmaceutically acceptable acid or base, optical isomers, mixtures, racemic mixtures, or any tautomeric form thereof.

14. The method according to claim 12, wherein the compound of formula (Ia) is 6-Chloro-3-(1-methylcyclopropyl)amino-4H-thieno[3,2-e]-1,2,4-thiadiazine 1,1-dioxide, a salt thereof with a pharmaceutically acceptable acid or base, optical isomers, mixtures, racemic mixtures, or any tautomeric form thereof.

15. The method according to claim 1, wherein the modulator of CD3 is administered in a regimen which additionally comprises administration of a beta cell resting compound.

16. The method according to claim 1, wherein the modulator of CD3 and the beta cell resting compound are co-administered.

17. A combination therapy which comprises at least one modulator of CD3 and at least one beta cell resting compound.

18. The combination according to claim 17 wherein the modulator of CD3 is a CD3 antibody or F(ab′)2 fragment thereof or other CD3 binding compound with same activity.

19. The combination according to claim 18, wherein the modulator of CD3 is anti-CD3 mAb OKT3 or F(ab′)2 fragment thereof.

20. The combination according to claim 18, wherein the modulator of CD3 is anti-CD3 mAb hOKT3γ1(Ala-Ala) or F(ab′)2 fragment thereof.

21. The combination according to claim 18, wherein the modulator of CD3 is anti-CD3 mAb 145 2C11 or F(ab′)2 fragment thereof.

22. The combination according to claim 18, wherein the modulator of CD3 is anti-CD3 mAb CAMPATH-3 or F(ab′)2 fragment thereof.

23. The combination according to claim 17 wherein the beta cell resting compound is a potassium channel opener.

24. The combination according to claim 23, wherein the beta cell resting compound is a compound of the general formula (I) 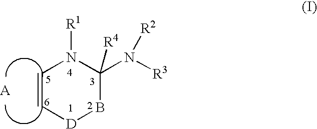
wherein
B represents >NR⁵ or >CR⁵R⁶, wherein R⁵ and R⁶ independently are hydrogen; hydroxy; C₁₋₆ alkoxy; or C₁₋₆-alkyl, C₃₋₆-cycloalkyl, C₂₋₆-alkenyl or C₂₋₆-alkynyl optionally mono- or polysubstituted with halogen; or R⁵ and R⁴ together represent one of the bonds in a double bond between the atoms 2 and 3 of formula (I);
D represents —S(═O)₂— or —S(═O)—; or
D-B represents —S(═O)(R⁷)═N—
wherein R⁷ is C₁₋₆-alkyl; or aryl or heteroaryl optionally mono- or polysubstituted with halogen, hydroxy, C₁₋₆-alkoxy, aryloxy, arylalkoxy, nitro, amino, C₁₋₆-monoalkyl- or dialkylamino, cyano, acyl, or C₁₋₆-alkoxycarbonyl;
R¹ is hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen and R⁴ is hydrogen; or R⁴ together with R⁵ represent one of the bonds in a double bond between the atoms 2 and 3 of formula (I); or R¹ together with R⁴ represent one of the bonds in a double bond between the atoms 3 and 4 of formula (I);
R² is hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen;
R³ is R⁸; —OR⁸; —C(═X)R⁸; —NR⁸R⁹; bicycloalkyl, aryl, heteroaryl, arylalkyl or heteroarylalkyl optionally mono- or poly substituted with halogen, hydroxy, C₁₋₆-alkoxy, aryloxy, arylalkoxy, nitro, amino, C₁₋₆-monoalkyl- or dialkylamino, cyano, oxo, acyl or C₁₋₆-alkoxycarbonyl; or aryl substituted with C₁₋₆-alkyl;
wherein R⁸ is hydrogen; C₃₋₆-cycloalkyl or (C₃₋₆-cycloalkyl)C₁₋₆-alkyl, the C₃₋₆-cycloalkyl group optionally being mono- or poly substituted with C₁₋₆-alkyl, halogen, hydroxy or C₁₋₆-alkoxy; a 3-6 membered saturated ring system comprising one or more nitrogen-, oxygen- or sulfur atoms; or straight or branched C₁₋₁₈-alkyl optionally mono- or poly substituted with halogen, hydroxy, C₁₋₆-alkoxy, C₁₋₆-alkylthio, C₃₋₆-cycloalkyl, aryl, aryloxy, arylalkoxy, nitro, amino, C₁₋₆-monoalkyl- or dialkylamino, cyano, oxo, formyl, acyl, carboxy, C₁₋₆-alkoxycarbonyl, or carbamoyl;
X is O or S;
R⁹ is hydrogen; C₁₋₆-alkyl; C₂₋₆-alkenyl; C₃₋₆-cycloalkyl optionally mono- or polysubstituted with C₁₋₆-alkyl, halogen, hydroxy or C₁₋₆-alkoxy; or
R⁸ and R⁹ together with the nitrogen atom form a 3-12 membered mono- or bicyclic system, in which one or more of the carbon atoms may be exchanged with nitrogen, oxygen or sulfur, each of these ring systems optionally being mono- or poly substituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino, oxo; or
R³ is 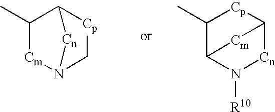
wherein n, m, p independently are 0,1,2,3 and R¹⁰ is hydrogen; hydroxy; C₁₋₆-alkoxy; C₃₋₆-cycloalkyl optionally mono- or poly substituted with C₁₋₆-alkyl, halogen, hydroxy or C₁₋₆-alkoxy; C₁₋₆-alkyl, C₂₋₆-alkenyl or C₂₋₆-alkynyl optionally mono- or polysubstituted with halogen; or
R² and R³ together with the nitrogen atom forms a 3-12 membered mono- or bicyclic system, in which one or more of the carbon atoms may be exchanged with nitrogen, oxygen or sulfur, each of these ring systems optionally being mono- or poly substituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆ monoalkyl- or dialkylamino or oxo;
A together with carbon atoms 5 and 6 of formula (I) represents a 5 or 6 membered heterocyclic system comprising one or more nitrogen-, oxygen- or sulfur atoms, the heterocyclic systems optionally being mono- or poly substituted with halogen; C₁₋₁₂-alkyl; C₃₋₆-cycloalkyl; hydroxy; C₁₋₆-alkoxy; C₁₋₆-alkoxy-C₁₋₆-alkyl; nitro; amino; cyano; cyanomethyl; perhalomethyl; C₁₋₆-monoalkyl- or dialkylamino; sulfamoyl; C₁₋₆-alkylthio; C₁₋₆-alkylsulfonyl; C₁₋₆-alkylsulfinyl; C₁₋₆-alkylcarbonylamino; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl; C₁₋₆-alkoxycarbonyl-C₁₋₆-alkyl; carbamyl; carbamyl-methyl; C₁₋₆-monoalkyl- or dialkylaminocarbonyl; C₁₋₆-monoalkyl- or dialkylaminothiocarbonyl; ureido; C₁₋₆-monoalkyl- or dialkylaminocarbonylamino, thioureido; C₁₋₆-monoalkyl- or dialkylaminothiocarbonyl-amino; C₁₋₆-monoalkyl- or dialkylaminosulfonyl; carboxy; carboxy-C₁₋₆-alkyl; acyl; aryl, arylalkyl, aryloxy, the aryl group optionally being mono- or polysubstituted with C₁₋₆-alkyl, halogen, hydroxy or C₁₋₆-alkoxy; (1,2,4-oxadiazol-5-yl)- or (1,2,4-oxadiazol-3-yl)-C₁₋₆-alkyl the oxadiazolyl group optionally being substituted with C₁₋₆-alkyl or C₃₋₆-cycloalkyl; or a 5-6 membered nitrogen containing ring, optionally substituted with phenyl or C₁₋₆-alkyl; or
a salt thereof with a pharmaceutically acceptable acid or base, optical isomers, mixtures, racemic mixtures, or any tautomeric form thereof.

25. The combination according to claim 24, wherein the compound of formula (I) is selected from the group:
6-Chloro-3-(1,2-dimethylpropyl)amino-4H-thieno[3,2-e]-1,2,4-thiadiazine 1,1-dioxide;
6-Chloro-3-ethylamino-4H-thieno[3,2-e]-1,2,4-thiadiazine 1,1-dioxide;
6-Chloro-3-isopropylamino-4H-thieno[3,2-e]-1,2,4-thiadiazine 1,1-dioxide;
(R)-6-Chloro-3-(1-phenylethyl)amino-4H-thieno[3,2-e]-1,2,4-thiadiazine 1,1-dioxide;
3-Allylamino-6-chloro-4H-thieno[3,2-e]-1,2,4-thiadiazine 1,1-dioxide;
6-Chloro-3-cyclopropylamino-4H-thieno[3,2-e]-1,2,4-thiadiazine 1,1-dioxide;
6-Chloro-3-hexylamino-4H-thieno[3,2-e]-1,2,4-thiadiazine 1,1-dioxide;
6-Chloro-3-tetradecylamino-4H-thieno[3,2-e]-1,2,4-thiadiazine 1,1-dioxide;
6-Chloro-3-methylamino-4H-thieno[3,2,e]-1,2,4-thiadiazine 1,1-dioxide;
3-Benzylamino-6-chloro-4H-thieno[3,2,e]-1,2,4-thiadiazine 1,1-dioxide;
6-Chloro-3-octylamino-4H-thieno[3,2-e]-1,2,4-thiadiazine 1,1-dioxide;
6-Chloro-3-isobutylamino-4H-thieno[3,2-e]-1,2,4-thiadiazine 1,1-dioxide;
6-Chloro-3-(4-phenylbutyl)amino-4H-thieno[3,2-e]-1,2,4-thiadiazine 1,1-dioxide;
6-Chloro-3-(1,5-dimethylhexyl)amino-4H-thieno[3,2-e]-1,2,4-thiadiazine 1,1-dioxide;
6-Chloro-3-propylamino-4H-thieno[3,2-e]-1,2,4-thiadiazine 1,1-dioxide;
(R)-6-Chloro-3-(2-hydroxy-1-methylethyl)amino-4H-thieno[3,2-e]-1,2,4-thiadiazine 1,1-dioxide;
(S)-6-Chloro-3-(2-hydroxy-1-methylethyl)amino-4H-thieno[3,2-e]-1,2,4-thiadiazine 1,1-dioxide;
(R)-3-sec-Butylamino-6-chloro-4H-thieno[3,2-e]-1,2,4-thiadiazine 1,1-dioxide;
3-Butylamino-6-chloro-4H-thieno[3,2-e]-1,2,4-thiadiazine 1,1-dioxide; and
3-Isopropylamino-7-methyl-4,7-dihydro-pyrazolo[4,3-e][1,2,4]thiadiazine 1,1-dioxide; or
a salt thereof with a pharmaceutically acceptable acid or base, optical isomers, mixtures, racemic mixtures, or any tautomeric form thereof.

26. The combination according to claim 24, wherein the compound of formula (i) is selected from the group:
3-Hydrazino-4H-pyrido[4,3-e]-1,2,4-thiadiazine 1,1-dioxide;
3-Benzylamino-4H-pyrido[4,3-e]-1,2,4-thiadiazine 1,1-dioxide;
3-(R)-(1-Phenylethylamino)-4H-pyrido[4,3-e]-1,2,4-thiadiazine 1,1-dioxide;
3-(S)-(1-Phenylethylamino)-4H-pyrido[4,3-e]-1,2,4-thiadiazine 1,1-dioxide;
3-Benzylamino-7-chloro-4H-pyrido[2,3-e]-1,2,4-thiadiazine 1,1-dioxide;
7-Chloro-3-(R)-(1-phenylethylamino)-4H-pyrido[2,3-e]-1,2,4-thiadiazine 1,1-dioxide;
7-Chloro-3-(S)-(1′-phenylethylamino)-4H-pyrido[2,3-e]-1,2,4-thiadiazine 1,1-dioxide;
3-Benzylamino-4H-pyrido[2,3-e]-1,2,4-thiadiazine 1,1-dioxide;
3-(R)-(1-Phenylethylamino)-4H-pyrido[2,3-e]-1,2,4-thiadiazine 1,1-dioxide;
3-(S)-(1-Phenylethylamino)-4H-pyrido[2,3-e]-1,2,4-thiadiazine 1,1-dioxide;
3-(Hexylamino)-4H-pyrido[4,3-e]-1,2,4-thiadiazine 1,1-dioxide;
7-Chloro-3-hexylamino-4H-pyrido[2,3-e]-1,2,4-thiadiazine 1,1-dioxide;
3-Octylamino-4H-pyrido[4,3-e]-1,2,4-thiadiazine 1,1-dioxide;
7-Chloro-3-octylamino-4H-pyrido[2,3-e]-1,2,4-thiadiazine 1,1-dioxide;
3-Allylamino-4H-pyrido[4,3-e]-1,2,4-thiadiazine 1,1-dioxide;
3-Allylamino-7-chloro-4H-pyrido[2,3-e]-1,2,4-thiadiazine 1,1-dioxide;
7-Chloro-3-(2-methoxy-1-methylethyl)amino-4H-pyrido[2,3-e]-1,2,4-thiadiazine 1,1-dioxide;
3-(2-Methoxy-1-methylethyl)amino-4H-pyrido[4,3-e]-1,2,4-thiadiazine 1,1-dioxide;
3-(2-Hydroxy-1-methylethyl)amino-4H-pyrido[4,3-e]-1,2,4-thiadiazine 1,1-dioxide;
3-Benzylamino-2-methyl-2H-pyrido[4,3-e]-1,2,4-thiadiazine 1,1-dioxide; and
2-Isopropylamino-3,3-dimethoxy-3H-pyrido[2,3-b][1,4]thiazine 4,4-dioxide; or
a salt thereof with a pharmaceutically acceptable acid or base, optical isomers, mixtures, racemic mixtures, or any tautomeric form thereof.

27. The combination according to claim 24, wherein the compound of formula (I) is selected from the following group:
7-Cyano-3-isopropylamino-6-methyl-4H-thieno[2,3-e]-1,2,4-thiadiazine 1,1-dioxide;
7-Cyano-6-methyl-3-propylamino-4H-thieno[2,3-e]-1,2,4-thiadiazine 1,1-dioxide;
6-Chloro-3-isopentylamino-4H-thieno[3,2-e]-1,2,4-thiadiazine 1,1-dioxide;
6-Chloro-3-(1-methylheptyl)amino-4H-thieno[3,2-e]-1,2,4-thiadiazine 1,1-dioxide;
6-Chloro-3-(1-ethylpentyl)amino-4H-thieno[3,2-e]-1,2,4-thiadiazine 1,1-dioxide;
6-Chloro-3-(2-methylbutyl)amino-4H-thieno[3,2-e]-1,2,4-thiadiazine 1,1-dioxide;
6-Chloro-3-(1-methylhexyl)amino-4H-thieno[3,2-e]-1,2,4-thiadiazine 1,1-dioxide;
6-Chloro-3-cyclopentylamino-4H-thieno[3,2-e]-1,2,4-thiadiazine 1,1-dioxide;
6-Chloro-3-cyclohexylmethylamino-4H-thieno[3,2-e]-1,2,4-thiadiazine 1,1-dioxide;
Ethyl 3-(6-chloro-1,4-dihydro-1,1-dioxothieno[3,2-e]-1λ⁶,2,4-thiadiazin-3-ylamino)-butanoate;
3-(6-Chloro-1,4-dihydro-1,1-dioxothieno[3,2-e]-1λ⁶,2,4-thiadiazin-3-ylamino)butanoic acid;
6-Chloro-3-(3-hydroxy-1-methylpropyl)amino-4H-thieno[3,2-e]-1,2,4-thiadiazine 1,1-dioxide;
(R)-6-Chloro-3-(1-phenylethyl)amino-4H-thieno[3,2-e]-1,2,4-thiadiazine 1,1-dioxide;
(S)-3-sec-Butylamino-6-chloro-4H-thieno[3,2-e]-1,2,4-thiadiazine 1,1-dioxide;
6-Chloro-3-isopropylamino-4H-thieno[2,3-e]-1,2,4-thiadiazine 1,1-dioxide;
6-Chloro-3-cyclopentylamino-4H-thieno[2,3-e]-1,2,4-thiadiazine 1,1-dioxide;
6-Bromo-3-isopropylamino-4H-thieno[3,2-e]-1,2,4-thiadiazine 1,1-dioxide;
3-Isopropylamino-4H-thieno[3,2-e]-1,2,4-thiadiazine 1,1-dioxide;
6-Fluoro-3-isopropylamino-4H-thieno[3,2-e]-1,2,4-thiadiazine 1,1-dioxide;
3-Cyclobutylamino-5,6-dimethyl-4H-thieno[3,2-e]-1,2,4-thiadiazine 1,1-dioxide;
3-Cyclopentylamino-5,6-dimethyl-4H-thieno[3,2-e]-1,2,4-thiadiazine 1,1-dioxide;
3-Isopropylamino-6,7-dimethyl-4H-thieno[2,3-e]-1,2,4-thiadiazine 1,1-dioxide;
3-Cyclobutylamino-6,7-dimethyl-4H-thieno[2,3-e]-1,2,4-thiadiazine 1,1-dioxide;
3-Cyclopentylamino-6,7-dimethyl-4H-thieno[2,3-e]-1,2,4-thiadiazine 1,1-dioxide;
5-Chloro-3-isopropylamino-4H-thieno[3,2-e]-1,2,4-thiadiazine 1,1-dioxide;
5-Chloro-3-propylamino-4H-thieno[3,2-e]-1,2,4-thiadiazine 1,1-dioxide;
5-Chloro-3-cyclopentylamino-4H-thieno[3,2-e]-1,2,4-thiadiazine 1,1-dioxide;
5-Chloro-6-methyl-3-isopropylamino-4H-thieno[3,2-e]-1,2,4-thiadiazine 1,1-dioxide;
6-chloro-3-isopropylamino-5-methyl-4H-thieno[3,2-e]-1,2,4-thiadiazine 1,1-dioxide;
6-chloro-3-cyclopentylamino-5-methyl-4H-thieno[3,2-e]-1,2,4-thiadiazine 1,1-dioxide;
6-Fluoro-3-propylamino-4H-thieno[3,2-e]-1,2,4-thiadiazine 1,1-dioxide;
6-Fluoro-3-cyclopentylamino-4H-thieno[3,2-e]-1,2,4-thiadiazine 1,1-dioxide;
5-Fluoro-3-propylamino-4H-thieno[3,2-e]-1,2,4-thiadiazine 1,1-dioxide;
5-Fluoro-3-isopropylamino-4H-thieno[3,2-e]-1,2,4-thiadiazine 1,1-dioxide;
3-Isopropylamino-7-methyl-4H-thieno[2,3-e]-1,2,4-thiadiazine 1,1-dioxide;
6-Chloro-3-cyclobutylamino-4H-thieno[3,2-e]-1,2,4-thiadiazine 1,1-dioxide;
6-Chloro-3-(2-hydroxyethyl)amino-4H-thieno[3,2-e]-1,2,4-thiadiazine 1,1-dioxide;
(±)-3-exo-Bicyclo[2.2.1]hept-2-ylamino-6-chloro-4H-thieno[3,2-e]-1,2,4-thiadiazine 1,1-dioxide;
(R)-6-Chloro-3-(2-hydroxypropyl)amino-4H-thieno[3,2-e]-1,2,4-thiadiazine 1,1-dioxide;
6-Bromo-3-isopropylamino-4H-thieno[3,2-e]-1,2,4-thiadiazine 1,1-dioxide;
5,6-Dibromo-3-isopropylamino-4H-thieno[3,2-e]-1,2,4-thiadiazine 1,1-dioxide;
6-Chloro-3-cyclohexylamino-4H-thieno[3,2-e]-1,2,4-thiadiazine 1,1-dioxide;
6-Chloro-3-(furan-2-ylmethyl)amino-4H-thieno[3,2-e]-1,2,4-thiadiazine 1,1-dioxide;
6-Chloro-3-(1-ethylpropyl)amino-4H-thieno[3,2-e]-1,2,4-thiadiazine 1,1-dioxide;
6-Bromo-3-cyclopentylamino-4H-thieno[3,2-e]-1,2,4-thiadiazine 1,1-dioxide;
6-Chloro-3-(2-methylallyl)amino-4H-thieno[3,2-e]-1,2,4-thiadiazine 1,1-dioxide; and
6-Cyano-3-isopropylamino-4H-thieno[3,2-e]-1,2,4-thiadiazine 1,1-dioxide; or
a salt thereof with a pharmaceutically acceptable acid or base, optical isomers, mixtures, racemic mixtures, or any tautomeric form thereof.

28. The combination according to claim 24, wherein the general formula (I) is 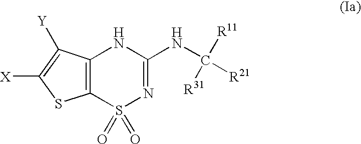
wherein
X and Y independently are hydrogen, halogen, perhalomethyl, C₁₋₆-alkyl or C₁₋₆-alkoxy;
R¹¹, R²¹ and R³¹ independently are C₁₋₆-alkyl, C₂₋₆-alkenyl, C₂₋₆-alkynyl, C₃₋₆-cycloalkyl, carboxy, C₁₋₆-alkoxycarbonyl or aryl, all of which are optionally being mono- or polysubstituted with halogen, hydroxy, oxo, or aryl; or
R¹¹ is as defined above and R²¹—C—R³¹ form a C₃₋₆-cycloalkyl group, optionally being mono- or polysubstituted with C₁₋₆-alkyl, perhalomethyl, halogen, hydroxy or aryl; or
—CR¹¹R²¹R³¹ form a 4- to 12-membered bicyclic or tricyclic carbocyclic system, optionally being mono- or polysubstituted with C₁₋₆-alkyl, perhalomethyl, halogen, hydroxy or aryl; or
a salt thereof with a pharmaceutically acceptable acid or base, optical isomers, mixtures, racemic mixtures, or any tautomeric form thereof.

29. The combination according to claim 28, wherein the compound of formula (Ia) is selected from the following group:
3-tert-Butylamino-6-chloro-4H-thieno[3,2-e]-1,2,4-thiadiazine 1,1-dioxide;
6-Chloro-3-(1,1-dimethylpropylamino)-4H-thieno[3,2-e]-1,2,4-thiadiazine 1,1-dioxide;
6-Chloro-3-(1-methylcyclopropyl)amino-4H-thieno[3,2-e]-1,2,4-thiadiazine 1,1-dioxide;
6-Chloro-3-(2-hydroxy-1,1-dimethylethylamino)-4H-thieno[3,2-e]-1,2,4-thiadiazine 1,1-dioxide;
6-Chloro-3-(1,1,3,3-tetramethylbutylamino)-4H-thieno[3,2-e]-1,2,4-thiadiazine 1,1-dioxide;
3-(1-Adamantyl)amino-6-chloro-4H-thieno[3,2-e]-1,2,4-thiadiazine 1,1-dioxide;
1-(6-Chloro-1,4-dihydro-1,1-dioxo-thieno[3,2-e]-l 16,2,4-thiadiazin-3-ylamino)-cyclopropanecarboxylic acid ethyl ester;
6-Chloro-3-(1-methyl-1-phenylethyl)amino-4H-thieno[3,2-e]-1,2,4-thiadiazine 1,1-dioxide;
6-Chloro-3-(1-hydroxymethylcyclopentyl)amino-4H-thieno[3,2-e]-1,2,4-thiadiazine 1,1-dioxide;
1-(6-Chloro-1,4-dihydro-1,1-dioxo-thieno[3,2-e]-1λ⁶,2,4-thiadiazin-3-ylamino)-cyclopropanecarboxylic acid;
6-Chloro-3-(1-methylcyclobutyl)amino-4H-thieno[3,2-e]-1,2,4-thiadiazine 1,1-dioxide;
6-Chloro-3-(1-methylcyclohexyl)amino-4H-thieno[3,2-e]-1,2,4-thiadiazine 1,1-dioxide;
6-Chloro-3-(1-methylcyclopentyl)amino-4H-thieno[3,2-e]-1,2,4-thiadiazine 1,1-dioxide; and
6-Chloro-3-(1-ethylcyclobutyl)amino-4H-thieno[3,2-e]-1,2,4-thiadiazine 1,1-dioxide; or
a salt thereof with a pharmaceutically acceptable acid or base, optical isomers, mixtures, racemic mixtures, or any tautomeric form thereof.

30. The combination according to claim 28, wherein the compound of formula (Ia) is 6-Chloro-3-(1-methylcyclopropyl)amino-4H-thieno[3,2-e]-1,2,4-thiadiazine 1,1-dioxide, a salt thereof with a pharmaceutically acceptable acid or base, optical isomers, mixtures, racemic mixtures, or any tautomeric form thereof.

31. A pharmaceutical composition comprising a combination of at least one modulator of CD3 and at least one beta cell resting compound
